(19) United States
(12) Patent Application Publication
(10) Pub. No.: US 2007/0077307 A1
(43) Pub. Date: Apr. 5, 2007

(54) PHARMACEUTICAL COMPOSITIONS

(76) Inventors: Joerg Rosenberg, Ellerstadt (DE); Katja Fastnacht, Mannheim (DE); Matthias Degenhardt, Ludwigshafen-Ruchheim (DE); Joerg Breitenbach, Mannheim (DE); Kennan C. Marsh, Lake Forest, IL (US); Rajeev Garg, Gurnee, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/422,741

(22) Filed: Jun. 7, 2006

Related U.S. Application Data
(63) Continuation of application No. 11/293,483, filed on Dec. 2, 2005.
(60) Provisional application No. 60/633,110.
(60) Provisional application No. 60/711,953, filed on Aug. 26, 2005.
(60) Provisional application No. 60/719,324, filed on Sep. 21, 2005.

(51) Int. Cl. A61K 31/235; A61K 9/14

(52) U.S. Cl. 424/489; 514/533

(57) ABSTRACT

The present invention relates to a pharmaceutical composition comprising primarily amorphous 2-[4-(4-chlorobenzoyl)phenoxy]-2-methyl-propanoic acid, 1-methylethyl ester.

### RELATED APPLICATION INFORMATION

This application is a continuation of U.S. application Ser. No. 11/293,483, filed on Dec. 2, 2005 which claims priority to U.S. Application No. 60/633,110, filed Dec. 3, 2004, U.S. Application No. 60/711,953 filed on Aug. 26, 2005 and U.S. Application No. 60/719,324 filed on Sep. 21, 2005, all of which are herein incorporated by reference.

### FIELD OF THE INVENTION

The present invention relates to a pharmaceutical composition comprising primarily amorphous 2-[4-(4-chlorobenzoyl)phenoxy]-2-methyl-propanoic acid, 1-methylethyl ester and a method of making same, as well as a method of treating dyslipidemia and dyslipoproteinemia by administering a therapeutically effective amount of said pharmaceutical composition to a subject in need thereof.

### BACKGROUND OF THE INVENTION

2-[4-(4-chlorobenzoyl)phenoxy]-2-methyl-propanoic acid, 1-methylethyl ester is a prodrug that is absorbed and then hydrolyzed by tissue and plasma esterases to 2-[4-(4-chlorobenzoyl)phenoxy]-2-methyl-propanoic acid (the active metabolite or active species). 2-[4-(4-chlorobenzoyl)phenoxy]-2-methyl-propanoic acid, 1-methylethyl ester is a benzophenone that contains a para-chlorophenyl and a para-isopropyloxycarbonylisopropoxyphenyl group. Both of these groups are substantially hydrophobic. Because of these hydrophobic groups, 2-[4-(4-chlorobenzoyl)phenoxy]-2-methyl-propanoic acid, 1-methylethyl ester is a poorly water soluble compound. In fact, 2-[4-(4-chlorobenzoyl)phenoxy]-2-methyl-propanoic acid, 1-methylethyl ester is practically insoluble in water. Because 2-[4-(4-chlorobenzoyl)phenoxy]-2-methyl-propanoic acid, 1-methylethyl ester is so poorly and variably absorbed, blood levels of active drug from an oral dose of 2-[4-(4-chlorobenzoyl)phenoxy]-2-methyl-propanoic acid, 1-methylethyl ester in a patient are susceptible to a food effect (meaning that there is variable uptake between fed and fasted states).

2-[4-(4-chlorobenzoyl)phenoxy]-2-methyl-propanoic acid, 1-methylethyl ester is marketed and prescribed for the treatment of dyslipidemia and dyslipoproteinemia. 2-[4-(4-chlorobenzoyl)phenoxy]-2-methyl-propanoic acid, 1-methylethyl ester has been made available commercially in a pharmaceutical dosage form (known as Lipidil®) which consists of a hard gelatin capsule containing crystalline 2-[4-(4-chlorobenzoyl)phenoxy]-2-methyl-propanoic acid, 1-methylethyl ester, lactose, pregelatinized starch and magnesium stearate. This formulation has been marketed in the United States as 200 mg and 67 mg capsules. After oral administration, during a meal, about 60% of the dose of this conventional formulation is effectively absorbed and found in the blood as 2-[4-(4-chlorobenzoyl)phenoxy]-2-methyl-propanoic acid (Weil et al., The metabolism and disposition of 14C-2-[4-(4-chlorobenzoyl)phenoxy]-2-methyl-propanoic acid, 1-methylethyl ester in human volunteers, Drug. Metabol. Dispos. Biol. Fate. Chem., 18:115-120 (1990)). 2-[4-(4-chlorobenzoyl)phenoxy]-2-methyl-propanoic acid lowers plasma triglycerides by potentially inhibiting triglyceride synthesis leading to a reduction of low density lipoprotein (LDL) released into the circulation. Measurement of the detected amount of 2-[4-(4-chlorobenzoyl)phenoxy]-2-methyl-propanoic acid in the blood of a patient can reflect the efficacy of 2-[4-(4-chlorobenzoyl)phenoxy]-2-methyl-propanoic acid, 1-methylethyl ester uptake.

Lipidil Micro®, as it is known outside the United States, is another pharmaceutical dosage form of 2-[4-(4-chlorobenzoyl)phenoxy]-2-methyl-propanoic acid, 1-methylethyl ester having improved bioavailability. European Patent Application 330,532 and U.S. Pat. No. 4,895,726 disclose a 2-[4-(4-chlorobenzoyl)phenoxy]-2-methyl-propanoic acid, 1-methylethyl ester composition in which crystalline 2-[4-(4-chlorobenzoyl)phenoxy]-2-methyl-propanoic acid, 1-methylethyl ester powder is micronized with a solid wetting agent and a process for making this composition. Sodium lauryl sulfate is described as the wetting agent of choice. The micronized powder is mixed with capsule filling excipients such as lactose, starch, cross-linked polyvinyl pyrrolidone (PVP), and magnesium stearate. A study comparing Lipidil Micro® to Lipidil® showed a statistically significant increase in bioavailability with the Lipidil Micro®. Lipidil Micro® has been marketed in the United States under the name TRICOR® (Micronized) as 160 mg and 54 mg tablets.

While Lipidil Micro® exhibits improved bioavailability, this composition does not lead to complete absorption of the dose of 2-[4-(4-chlorobenzoyl)phenoxy]-2-methyl-propanoic acid, 1-methylethyl ester and suffers from several disadvantages. Specifically, while bioavailability of 2-[4-(4-chlorobenzoyl)phenoxy]-2-methyl-propanoic acid is improved in Lipidil Micro®, the formulation remains subject to differences in bioavailability when taken with a meal or in the fasted state.

To date, there are no pharmaceutical compositions that are available that contain primarily amorphous 2-[4-(4-chlorobenzoyl)phenoxy]-2-methyl-propanoic acid, 1-methylethyl ester and have no significant food effect upon oral administration. In view of this, it is an object of the present invention to provide a pharmaceutical composition that can be orally administered and upon dissolution provides a suspension comprising particles of 2-[4-(4-chlorobenzoyl)phenoxy]-2-methyl-propanoic acid, 1-methylethyl ester. The pharmaceutical composition of the present invention exhibits enhanced bioavailability when compared to a reference formulation (as defined herein) and provides a formulation that has no significant food effect.

### SUMMARY OF THE PRESENT INVENTION

In one embodiment, the present invention relates to an oral pharmaceutical composition comprising at least one active agent, wherein the active agent comprises primarily amorphous 2-[4-(4-chlorobenzoyl)phenoxy]-2-methyl-propanoic acid,1-methylethyl ester. The oral composition of the present invention lacks a significant food effect on oral administration.

The 2-[4-(4-chlorobenzoyl)phenoxy]-2-methyl-propanoic acid,1-methylethyl ester is present in the composition in an amount of from about 5 weight percent to about 65 weight percent of the total composition, specifically, from about 10 weight percent to about 50 weight percent of the total composition.

In addition to the primarily amorphous 2-[4-(4-chlorobenzoyl)phenoxy]-2-methyl-propanoic acid, 1-methylethyl ester, the composition of the present invention further comprises at least one pharmaceutically acceptable polymer and, optionally, at least one pharmaceutically acceptable surfactant. The composition of the present invention can also contain at least one solubility-enhancing agent. The composition of the present invention can also contain at least one coating, tableting aids, water-soluble polymers, fillers, binders, pigments, distintegrants, antioxidants, lubricants, flow aids and/or flavorants.

The at least one pharmaceutically acceptable polymer can be present in the composition in an amount of from about 20 weight percent to about 95 weight percent, preferably, from about 30 weight percent to about 75 weight percent. The at least one pharmaceutically acceptable polymer that can be used in the composition can be an ionic cellulosic polymer. For example, the ionic cellulosic polymer includes, but is not limited to, carboxymethylcellulose (CMC), carboxymethylcellulose (CMC) salts, such as, but not limited to, carboxymethylcellulose sodium salts, carboxyethylcellulose (CEC), hydroxyethylmethylcellulose acetate phthalate, hydroxyethylmethylcellulose acetate succinate, hydroxypropylmethylcellulose phthalate (HPMCP), hydroxypropylmethylcellulose succinate, hydroxypropylcellulose acetate phthalate (HPCAP), hydroxypropylcellulose acetate succinate (HPCAS), hydroxypropylmethylcellulose acetate phthalate (HPMCAP), hydroxypropylmethylcellulose acetate succinate (HPMCAS), hydroxypropylmethylcellulose acetate trimellitate (HPMCAT), hydroxypropylmethylcellulose acetate phthalate (HPMCAP), hydroxypropylcellulose butyrate phthalate, carboxymethylethylcellulose and salts thereof, such as, but not limited to sodium salts of carboxymethylethylcellulose, cellulose acetate phthalate (CAP), methylcellulose acetate phthalate, cellulose acetate trimellitate (CAT), cellulose acetate terephthalate, cellulose acetate isophthalate, cellulose propionate phthalate, cellulose propionate trimellitate, cellulose butyrate trimellitate and combinations thereof.

Alternatively, the at least one pharmaceutically acceptable polymer can be a nonionic cellulosic polymer. For example, the nonionic cellulosic polymer includes, but is not limited to, methylcellulose, ethyl cellulose, hydroxyethyl cellulose, hydroxypropylcellulose, hydroxypropylmethylcellulose, hydroxypropylmethylcellulose acetate, hydroxyethylmethylcellulose, hydroxyethylcellulose acetate, hydroxyethylethylcellulose and combinations thereof.

Alternatively, the pharmaceutically acceptable polymer can be methyacrylic acid copolymers, aminoalkyl methacrylate copolymers, carboxylic acid functionalized polymethacrylates, amine-functionalized polymethacrylates, poly(vinyl acetal) diethylaminoacetate, polyvinyl pyrrolidone, polyvinyl alcohol, polyvinyl alcohol/polyvinyl acetate copolymers and combinations thereof.

More specifically, the pharmaceutically acceptable polymer can be polyvinyl pyrrolidone, polyvinyl alcohol/polyvinyl acetate copolymers and combinations thereof.

Alternatively, the pharmaceutically acceptable polymer can be polyethylene oxide polyethylene glycol/polypropylene glycol copolymers, polyethylene/polyvinyl alcohol copolymers, dextran, pullulan, acacia, tragacanth, sodium alginate, propylene glycol alginate, agar powder, gelatin, starch, processed starch, glucomman, chitosan and combinations thereof.

In addition, mixtures of pharmaceutically acceptable polymers can be utilized in the present invention. The polymers may be selected to modulate the hydrophilicity of the pharmaceutical composition.

If the pharmaceutical composition contains at least one pharmaceutically acceptable surfactant, said surfactant can have a hydrophile-lipophile balance (HLB) value from about 1 to about 20. The at least one pharmaceutically acceptable surfactant can be present in the composition in an amount of from about 0.5 weight percent to about 20 weight percent, preferably, from about 1 weight percent to about 8 weight percent. The at least one pharmaceutically acceptable surfactant that can be used in the composition in the present invention, includes, but is not limited to, triglycerides of caprylic/capric acid, propylene glycol laurate, glyceryl and polyethylene glycol esters, sorbitan monooleate, sorbitan monolaurate, mono or diglycerides of caprylic/capric acid in glycerol, sorbitan sesquioleate, polyoxyethylene (2) oleyl ether, polyoxypropylene 15 stearyl ether, unsaturated polyglycolyzed glycerides, glyceryl monolinoleate, decaglyceryl decaoleate, triisostearin polyethylene glycol 6 esters, triglyceryl monoleate, glyceryl monooleate, sorbide dioleate, polyoxyethylene castor wax, polyglycolysed glycerides, polyglycolysed glycerides, saturated C₈-C₁₀ polyglycolysed glycerides, polyoxyethlene (20) sorbitan monooleate, polyoxyethylene (20) sorbitan trioleate, copolymers of propylene oxide and ethylene oxide, polyoxyl 35 castor oil, palm kernelamide, polyoxyethylene 4 lauryl ether, polyoxyethylene (20) isohexadecyl ether, sorbitan monolaurate, alcohol ethoxylate, polyoxyethylene 80 sorbitan monolaurate, hexaglyceryl dioleate, polysorbate 80, sucrose laurate, quaternary ammonium salt, polyoxyethylene sorbitol hexaoleate, caprylic/capric acid partial glyceride-6 EO, polyglyceryl 4 oleate, and combinations thereof.

If the composition of the present invention contains at least one solubility-enhancing agent, said agent is present in the composition in the amount of from about 1 weight percent to about 40 weight percent, preferably, from about 1 weight percent to about 10 weight percent. Examples of solubility-enhancing agents that can be used in the composition of the present invention include at least one surfactant, at least one pH control agent, glycerides, partial glycerides, glyceride derivatives, polyoxyethylene and polypropylene esters and copolymers, sorbitan esters, polyoxyethylene sorbitan esters, carbonate salts, alkyl sulfonates, cyclodextrins and combinations thereof.

The pharmaceutical composition of the present invention can be in the form of a solid dispersion. When said solid dispersion is placed in contact with an aqueous medium, said solid dispersion forms a suspension comprising particles containing 2-[4-(4-chlorobenzoyl)phenoxy]-2-methyl-propanoic acid, 1-methylethyl ester. The D₅₀ of the particles in the suspension are from a D₅₀ of about 1 μm to a D₅₀ of about 100 μm. The particles can contain crystalline 2-[4-(4-chlorobenzoyl)phenoxy]-2-methyl-propanoic acid, 1-methylethyl ester, amorphous 2-[4-(4-chlorobenzoyl)phenoxy]-2-methyl-propanoic acid, 1-methylethyl ester or a mixture of crystalline and amorphous 2-[4-(4-chlorobenzoyl)phenoxy]-2-methyl-propanoic acid, 1-methylethyl ester. The suspension comprising particles containing 2-[4-(4-chlorobenzoyl)phenoxy]-2-methyl-propanoic acid, 1-methylethyl ester has improved bioavailability compared to a reference formulation (namely, a 200 mg or 67 mg oral capsule pharmaceutical composition comprising crystalline 2-[4-(4-chlorobenzoyl)phenoxy]-2-methyl-propanoic acid,1-methylethyl ester).

In another embodiment, the present invention relates to a method of treating dyslipidemia in a subject in need of treatment thereof by administering to said subject a therapeutically effective amount of the hereinbefore described pharmaceutical composition.

In yet another embodiment, the present invention relates to a method of treating dyslipoproteinemia in a subject in need of treatment thereof by administering to said subject a therapeutically effective amount of the hereinbefore described pharmaceutical composition.

### DESCRIPTION OF THE FIGURES

FIG. 1 shows six (6) different suspensions containing 2-[4-(4-chlorobenzoyl)phenoxy]-2-methyl-propanoic acid, 1-methylethyl ester.

FIG. 2 is a graph demonstrating the mean plasma concentration of 2-[4-(4-chlorobenzoyl)phenoxy]-2-methyl-propanoic acid after an approximately 54 mg single orally administered dose of 2-[4-(4-chlorobenzoyl)phenoxy]-2-methyl-propanoic acid, 1-methylethyl ester in fasted dogs.

FIG. 3 shows the differential scanning calorimetry for solid dispersions 1-0, 1-5, 1-8, 1-13 and 1-16 as described in Example 6.

FIGS. 4A-4E shows three (3) samples of each of seven (7) different suspensions containing 2-[4-(4-chlorobenzoyl)phenoxy]-2-methyl-propanoic acid, 1-methylethyl ester at various time points, specifically, immediately after formation of the suspension (“initial”), at 6 hours after initial formation, 1 day after initial formation, 3 days after initial formation and 7 days after initial formation.

FIGS. 5A-5D shows the dispersion characterization of each of the suspensions shown in FIG. 4 as determined immediately after formation (namely, “initial”), 1 day after initial formation, 3 days after initial formation and 7 days after initial formation.

### DETAILED DESCRIPTION OF THE INVENTION

I. Definitions

As used in this specification and the appended claims, the singular forms “a,” “an” and “the” include plural references unless the context clearly dictates otherwise. Thus, for example, reference to “an active agent” includes a single active agent as well two or more different active agents in combination, reference to “an excipient” includes mixtures of two or more excipients as well as a single excipient, and the like.

In describing and claiming the present invention, the following terminology will be used in accordance with the definitions set out below.

The term “AUC” refers to the area under the plasma concentration time curve and is calculated by the trapezoidal rule. The term “AUCO₀₋ₜ” means the area under the plasma concentration curve from time 0 to the last measurable concentration in units of μg·h/mL as determined using the trapezoidal rule. The term “AUC_{0-∞}” means the area under the plasma concentration curve from time 0 to infinite time. AUC(_{0-∞}) is calculated as AUC(₀₋ₜ)+LMT/(−β), where “LMT” is the last measurable plasma concentration and β is the terminal phase elimination rate constant.

The terms “active agent,” “pharmacologically active agent,” and “drug” are used interchangeably herein to refer to 2-[4-(4-chlorobenzoyl)phenoxy]-2-methyl-propanoic acid, 1-methylethyl ester. The terms also encompass analogs of −[4-(4-chlorobenzoyl)phenoxy]-2-methyl-propanoic acid, 1-methylethyl ester. When the terms “active agent,” “pharmacologically active agent” and “drug” are used, it is to be understood that Applicants intend to include 2-[4-(4-chlorobenzoyl)phenoxy]-2-methyl-propanoic acid, 1-methylethyl esterper se as well as analogs of 2-[4-(4-chlorobenzoyl)phenoxy]-2-methyl-propanoic acid, 1-methylethyl ester.

As used herein, the term the “bioavailability” when used in connection with a composition or compound is synonymous with the “AUC” of the composition or compound when compared against a reference composition or compound.

The term “Cₘₐₓ” refers to the maximum observed plasma concentration of 2-[4-(4-chlorobenzoyl)phenoxy]-2-methyl-propanoic acid produced by the ingestion of the compositions of the present invention.

The terms “dyslipidemia” and “dyslipoproteinemia” as used herein, include the conditions in the group selected from hypercholesterolemia, abnormal and elevated levels of cholesterol, abnormal and elevated levels of LDL cholesterol, abnormal and elevated levels of total cholesterol, abnormal and elevated levels of plasma cholesterol, abnormal and elevated levels of triglycerides, hypertrigylceridaemia, abnormal levels of lipoproteins, abnormal and elevated levels of low density lipoproteins (LDLs), abnormal and elevated levels of very low density lipoproteins, abnormal and elevated levels of very low intermediate density lipoproteins, abnormal levels of high density lipoproteins, hyperlipidemia, hyperchylomicronemia, abnormal levels of chylomicrons, related disorders, and combinations thereof such as those described in The ILIB Lipid Handbook for Clinical Practice, Blood Lipids and Coronary Heart Disease, Second Edition, A. M. Gotto et al, International Lipid Information Bureau, New York, N.Y., 2000, which is hereby incorporated by reference. Elevation of serum cholesterol, triglyercides, or both is characteristic of hyperlipidemias. Differentiation of specific abnormalities usually requires identification of specific lipoprotein fractions in the serum of a patient. Lipoproteins transport serum lipids and can be identified by their density and electrophoretic mobility. Chylomicrons are among the largest and least dense of the lipoproteins. Others, in order of increasing density and decreasing size include very low density lipoproteins (VLDL or pre-beta), intermediate low density lipoproteins (ILDL or broad-beta), low density lipoproteins (LDL or beta), and high density lipoproteins (HDL or alpha). Triglycerides are transported primarily by chylomicrons and very low density lipoproteins. Cholesterol is transported primarily by low density lipoproteins. Hyperlipidemia types include type I, typeIIa, type IIb, type III, type IV, and type V. These types can be characterized according to the levels relative to normal of lipids (cholesterol and triglycerides) and lipoproteins described above.

The terms “treating” and “treatment” refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease.

The term “Tₘₐₓ” refers to the time to the maximum observed plasma concentration of 2-[4-(4-chlorobenzoyl)phenoxy]-2-methyl-propanoic acid produced by the ingestion of the formulations of the present invention.

By an “effective amount” or a “therapeutically effective amount” of an active agent is meant a nontoxic but sufficient amount of the active agent to provide the desired effect. The amount of active agent that is “effective” will vary from subject to subject, depending on the age and general condition of the individual, the particular active agent or agents, and the like. Thus, it is not always possible to specify an exact “effective amount.” However, an appropriate “effective amount” in any individual case may be determined by one of ordinary skill in the art using routine experimentation.

The phrases, “fasted patient”, “fasting patient”, “fasting conditions” or “fasting” refers to a patient who has not eaten any food, i.e., who has fasted for at least 10 hours before the administration of the oral formulation of the present invention comprising primarily amorphous 2-[4-(4-chlorobenzoyl)phenoxy]-2-methyl-propanoic acid, 1-methylethyl ester and analogs of 2-[4-(4-chlorobenzoyl)phenoxy]-2-methyl-propanoic acid, 1-methylethyl ester and who does not eat any food and continues to fast for at least 4 hours after the administration of the formulation. The formulation is preferably administered with 240 ml of water during the fasting period, and water can be allowed ad libitum up to 1 hour before and 1 hour after ingestion.

The phrases a “fed patient”, “fed conditions” or “fed” refer to a patient who has fasted for at least 10 hours overnight and then has consumed an entire test meal beginning 30 minutes before the first ingestion of the test formulations. The formulation of the present invention is administered with 240 ml of water within 5 minutes after completion of the meal. No food is then allowed for at least 4 hours post-dose. Water can be allowed ad libitum up to 1 hour before and 1 hour after ingestion. A high fat test meal provides approximately 1000 calories to the patient of which approximately 50% of the caloric content is derived from fat content of the meal. A representative high fat high calorie test meal comprises 2 eggs fried in butter, 2 strips of bacon, 2 slices of toast with butter, 4 ounces of hash brown potatoes and 8 ounces of whole milk to provide 150 protein calories, 250 carbohydrate calories and 500 to 600 fat calories. High fat meals can be used in clinical effect of food studies of 2-[4-(4-chlorobenzoyl)phenoxy]-2-methyl-propanoic acid, 1-methylethyl ester. A low fat test meal provides approximately 600 calories to the patient of which approximately 30% of the caloric content is derived from fat content of the meal.

The term “suspension” refers to particles dispersed in an aqueous medium wherein the particles of such suspension are preferably solid and comprise 2-[4-(4-chlorobenzoyl)phenoxy]-2-methyl-propanoic acid, 1-methylethyl ester, the particles having a size between about 100 μm and about 500 μm, preferably between about 1 nm and about 100 μm.

The phrases “positive food effective” or “food effect” refer to when the amount of an active agent or drug taken into the blood from a given oral composition or dosage form by a fasting patient is less than the amount of the active drug taken into the blood from the same oral composition or dosage form by the same patient who has been fed a high fat containing meal proximal to the time of administration of the oral composition or dosage form.

By “pharmaceutically acceptable,” such as in the recitation of a “pharmaceutically acceptable excipient,” or a “pharmaceutically acceptable additive,” is meant a material that is not biologically or otherwise undesirable, i.e., the material may be incorporated into a pharmaceutical composition administered to a patient without causing any undesirable biological effects.

As used herein, the term “reference formulation” refers to an oral capsule dosage form containing either 200 mg or 67 mg of conventional microcrystalline 2-[4-(4-chlorobenzoyl)phenoxy]-2-methyl-propanoic acid, 1-methylethyl ester. The reference formulation has been marketed as Lipidil®.

The term “solid dispersion” refers to an active agent or drug dispersed or dissolved in a vehicle, carrier, diluent or matrix in the solid state. For example, the active agent or drug may be dispersed or dissolved in at least one pharmaceutically acceptable polymer, at least one pharmaceutically acceptable surfactant, a mixture of at least one pharmaceutically acceptable polymer and at least one pharmaceutically acceptable surfactant, etc.

The term “subject” refers to an animal, preferably a mammal, including a human or non-human. The terms patient and subject may be used interchangeably herein.

The Present Invention

The present invention relates to oral pharmaceutical compositions that comprise at least one active agent, wherein at least one active agent is primarily amorphous, namely, in a non-crystalline state, 2-[4-(4-chlorobenzoyl)phenoxy]-2-methyl-propanoic acid, 1-methylethyl ester. The pharmaceutical compositions of the present invention, upon contact with an aqueous medium, such as that found in the gastrointestinal tract of a subject, form a suspension that contains particles that comprise 2-[4-(4-chlorobenzoyl)phenoxy]-2-methyl-propanoic acid, 1-methylethyl ester. The particles contained within such a suspension typically have a particle size of from about 100 nm (0.1 microns) to about 500 microns, preferably from about 1 micron to about 100 microns. The particles can contain crystalline 2-[4-(4-chlorobenzoyl)phenoxy]-2-methyl-propanoic acid, 1-methylethyl ester, amorphous 2-[4-(4-chlorobenzoyl)phenoxy]-2-methyl-propanoic acid, 1-methylethyl ester or a mixture of crystalline and amorphous 2-[4-(4-chlorobenzoyl)phenoxy]-2-methyl-propanoic acid, 1-methylethyl ester. The suspension is stable in water, meaning that the suspension does not coagulate. Particles in the suspensions that are not stable will coagulate and possibly even form an agglomerate. In addition, when administered to a subject orally, the compositions of the present invention lack a significant food effect.

In one embodiment, the oral pharmaceutical compositions of the present invention are made by first preparing a solid dispersion comprising primarily amorphous 2-[4-(4-chlorobenzoyl)phenoxy]-2-methyl-propanoic acid, 1-methylethyl ester, at least one pharmaceutically acceptable polymer and optionally, at least one pharmaceutically acceptable surfactant.

Prior to the formation of the solid dispersion, the 2-[4-(4-chlorobenzoyl)phenoxy]-2-methyl-propanoic acid, 1-methylethyl ester, in its pure state, can be either amorphous or crystalline. In other words, the form of 2-[4-(4-chlorobenzoyl)phenoxy]-2-methyl-propanoic acid, 1-methylethyl ester, either amorphous or crystalline, prior to the formation of the solid dispersion is not critical. If crystalline 2-[4-(4-chlorobenzoyl)phenoxy]-2-methyl-propanoic acid, 1-methylethyl ester is used, it can be converted into amorphous 2-[4-(4-chlorobenzoyl)phenoxy]-2-methyl-propanoic acid, 1-methylethyl ester when the solid dispersion is prepared. Techniques for preparing such a solid dispersion are described in more detail herein.

Solid dispersions comprising amorphous 2-[4-(4-chlorobenzoyl)phenoxy]-2-methyl-propanoic acid, 1-methylethyl ester can be prepared using techniques known to those skilled in the art, such as, but not limited to, melt extrusion, evaporation, curing, microwaves, milling, ultra sound, spinning disc, etc. Such methods are disclosed in, e.g., U.S. Pat. No. 4,880,585, U.S. Pat. No. 6,254,889, U.S. Pat. No. 6,387,401, U.S. Pat. No. 6,706,283, U.S. Pat. No. 6,599,528, U.S. Pat. No. 5,546,923, U.S. Patent Application No. 2004/0013697, JP-A 58-192817 and JP-A 58-79915. However, when the 2-[4-(4-chlorobenzoyl)phenoxy]-2-methyl-propanoic acid, 1-methylethyl ester is dispersed or dissolved in at least one pharmaceutically acceptable polymer for making said solid dispersion, the 2-[4-(4-chlorobenzoyl)phenoxy]-2-methyl-propanoic acid, 1-methylethyl ester should primarily (e.g., about 80% or greater) be in an amorphous state (in other words, it should be “primarily amorphous”), such that its predominantly non-crystalline nature is identifiable by techniques known in the art, e.g., X-ray diffraction analysis or by differential scanning calorimetry. The solid dispersion can contain a small amount of the 2-[4-(4-chlorobenzoyl)phenoxy]-2-methyl-propanoic acid, 1-methylethyl ester (e.g. about 20% or less) in a crystalline state. The solid dispersion may contain from about 5% to about 65% by weight of 2-[4-(4-chlorobenzoyl)phenoxy]-2-methyl-propanoic acid, 1-methylethyl ester, preferably from about 10% to about 50% by weight of 2-[4-(4-chlorobenzoyl)phenoxy]-2-methyl-propanoic acid, 1-methylethyl ester, and more preferably from about 10% to about 30% by weight of 2-[4-(4-chlorobenzoyl)phenoxy]-2-methyl-propanoic acid, 1-methylethyl ester.

With respect to the at least one pharmaceutically acceptable polymer to be used in the solid dispersion, any polymer that can be used with 2-[4-(4-chlorobenzoyl)phenoxy]-2-methyl-propanoic acid, 1-methylethyl ester (amorphous or crystalline) and is a pharmaceutically acceptable polymer can be used to form the dispersion. More specifically, polymers that can be used can be virtually any natural or synthetic polymer that can be used as a raw material in the manufacture of a pharmaceutical composition. For example, polymers that can be used include pH-sensitive polymers, water-soluble polymers, etc. The amount of the polymer present in the dispersion generally ranges from about 20 wt % to about 95 wt % and preferably from about 50 wt % to about 90 wt %. The choice of polymer to be selected for use in the solid dispersion may depend upon the technique to be used for making said dispersion. Moreover, polymers can be used independently or, if necessary, in combinations of two or more.

Polymers that can be used in the dispersion include ionizable and nonionizable cellulosic polymers (including those with ether or ester or a mixture of ester/ether substituents and copolymers thereof, including both so-called “enteric” and “non-enteric” polymers); and vinyl polymers and copolymers having substituents of hydroxyl, alkylacyloxy and cyclicamido.

Exemplary ionic cellulosic polymers include, but are not limited to, carboxymethylcellulose (CMC) and salts thereof, such as, but not limited to, sodium salts of carboxymethylcellulose, carboxyethylcellulose (CEC), hydroxyethylmethylcellulose acetate phthalate, hydroxyethylmethylcellulose acetate succinate, hydroxypropylmethylcellulose phthalate (HPMCP), hydroxypropylmethylcellulose succinate, hydroxypropylcellulose acetate phthalate (HPCAP), hydroxypropylcellulose acetate succinate (HPCAS), hydroxypropylmethylcellulose acetate phthalate (HPMCAP), hydroxypropylmethylcellulose acetate succinate (HPMCAS), hydroxypropylmethylcellulose acetate trimellitate (HPMCAT), hydroxypropylmethylcellulose acetate phthalate (HPMCAP), hydroxypropylcellulose butyrate phthalate, carboxymethylethylcellulose and salts thereof, such as, but not limited to, sodium salts of carboxymethylethylcellulose, cellulose acetate phthalate (CAP), methylcellulose acetate phthalate, cellulose acetate trimellitate (CAT), cellulose acetate terephthalate, cellulose acetate isophthalate, cellulose propionate phthalate, cellulose propionate trimellitate, cellulose butyrate trimellitate and mixtures thereof.

Exemplary nonionic cellulosic polymers include, but are not limited to, methylcellulose (MC), ethyl cellulose (EC), hydroxyethyl cellulose (HEC), hydroxypropylcellulose (HPC), hydroxypropylmethylcellulose (HPMC), hydroxypropylmethylcellulose acetate, hydroxyethylmethylcellulose, hydroxyethylcellulose acetate, hydroxyethylethylcellulose and mixtures thereof.

Exemplary vinyl polymers and copolymers include, but are not limited to, methacrylic acid copolymers, aminoalkyl methacrylate copolymers, carboxylic acid functionalized polymethacrylates, and amine-functionalized polymethacrylates, poly(vinyl acetal) diethylaminoacetate, polyvinyl pyrrolidone (PVP), copovidone, polyvinyl alcohol (PVA), polyvinyl alcohol/polyvinyl acetate (PVA/PVAc) copolymers and mixtures thereof. Polyvinyl pyrrolidone (PVP) and polyvinyl alcohol/polyvinyl acetate (PVA/PVAc) copolymers are preferred.

Other polymers that can be used include, but are not limited to, polyethyleneoxide polyethylene glycol/polypropylene glycol (PEG/PPG) copolymers, polyethylene/polyvinyl alcohol (PE/PVA) copolymers, dextran, pullulan, acacia, tragacanth, sodium alginate, propylene glycol alginate, agar powder, gelatin, starch, processed starch, glucomannan, chitosan and mixtures thereof.

As mentioned briefly above, the solid dispersion can optionally contain at least one pharmaceutically acceptable surfactant. Suitable surfactants will typically be those with hydrophile-lipophile balance (“HLB”) values ranging from about 1 to about 20 and present in an amount of about 0.5 wt % to about 20 wt %, and preferably from about 1 wt % to about 8 wt %.

Exemplary surfactants include, but are not limited to, Labrafac® Lipophile WL 1349 (triglyceride of caprylic/capric acid; Gattefosse, Ltd., Great Britain (hereinafter “Gattefosse”), Lauroglycol® FCC (propylene glycol laurate; Gattefosse), Labrafil® M 1944 CS (glyceryl and polyethylene glycol esters; Gattefosse), Span® 80 (sorbitan monooleate; Sigma), Span 20® (sorbitan monolaurate), Capmul® MCM (mono/diglycerides of caprylic/capric acid in glycerol; Abitec), Arlacel® 83 (sorbitan sesquioleate; ICI), Brij® 93 (polyoxyethylene (2) oleyl ether; READ ICI), Acconon® E (polyoxypropylene 15 stearyl ether; Abitec), Labrafil® M 2125 CS (unsaturated polyglycolyzed glycerides; Gattefose), Maisine 35-1 (glyceryl monolinoleate; Gattefosse), Sorbitan Oleate NF (Crill #4; Croda), Caprol® 10G100 (decaglyceryl decaoleate; Abitec), Labrafil® Isostearique® (triisostearin PEG 6 esters; Gattefosse), Caprol® 3G0 triglyceryl monoleate; Abitec), Peceol® (glyceryl monooleate; Gattefosse), G-950 (sorbide dioleate; ICI), Arlacel® 989 (polyoxyethylene castor wax; ICI), Labrafac® CM 10 (polyglycolysed glycerides; Gattefosse), Labrafac® CM 12 (polyglycolysed glycerides; Gattefosse), Labrasol® (saturated C₈-C₁₀ polyglycolysed glycerides; Gattefosse), Tween® 80 (polyoxyethylene (20) sorbitan monooleate; Sigma), Tween® 85 (polyoxyethylene (20) sorbitan trioleate; Sigma) Tween 20, Pluronic® L43 (copolymers of propylene oxide and ethylene oxide; BASF), Pluronic® 17R4 (copolymers of propylene oxide and ethylene oxide; BASF), Cremophor® EL (polyoxyl 35 castor oil; BASF), Accomid® PK (palm kernelamide DEA; Abitec), Brij® 30 (polyoxyethylene 4 lauryl ether; READ ICI), Arlasolve 200 liquid (polyoxyethylene (20) isohexadecyl ether; ICI), Arlacel® 20 (sorbitan monolaurate; ICI), Renex® 38 (alcohol ethoxylate; ICI), G-4280 (polyoxyethylene 80 sorbitan monolaurate; ICI), Caprol® 6G20 (hexaglyceryl dioleate; Abitec), Crillet® 4 Ultra (polysorbate 80; Croda), Crodesta® SL-40 (sucrose laurate; Croda), Cirrasol® G-265 (quaternary ammoniun salt; ICI), Cirrasol G-1096 (polyoxyethylene sorbitol hexaoleate; ICI), Softigen® 767 (caprylic/capric acid partial glyceride-6 EO; HULS America), Witconol® 14 (polyglyceryl 4 oleate; Witco), Miglyol® (HULS America) and combinations of one or more of the above surfactants.

Examples of preferred surfactants include Labrafil® M 1944 CS (glyceryl and polyethylene glycol esters; Gattefosse), Span 20® (sorbitan monolaurate), Tween® 85 (polyoxyethylene (20) sorbitan trioleate; Sigma), Cremophor® RH-40 (polyoxyl 35 hydrogenated castor oil; BASF), Miglyol® and combinations of these surfactants, particularly, Miglyol® and Cremophor® RH-40.

Suitable oils that can be used as surfactants include, but are not limited to, any pharmaceutically acceptable oil, such as, for example, Labrafac®, Lipophile WL 1349 (triglyceride of caprylic/capric acid; Gattefosse), Myvacet® 9-08 (distillated acetylated monoglycerides), Myvacet® 9-40 (distillated acetylated monoglycerides), Capmul® PG-8 (propylene glycol and mono/di-caprylate; Abitec), Arlamol® E (polyoxypropylene (15) stearyl alcohol; ICI), Captex® 300 (glyceryl tricaprylate/caprate; Abitec), olive oil, Miglyol® 812 (caprylic/capric triglycerides; HULS America), sesame oil (Sigma), Novol® (oleyl alcohol, Croda). Preferred oils include Labrafac®, Lipophile® WL 1349, Myvacet® 9-08, Myvacet® 9-40, Capmul® PG-8 and combinations thereof.

The pharmaceutical composition of the present invention can optionally include solubility-enhancing agents that promote the water solubility of the active agent. Such solubility-enhancing agents can be present in an amount ranging from about 1 wt % to about 40 wt %, and preferably from about 1 wt % to about 10 wt % of the total weight of the formulation. Examples of suitable solubility-enhancing agents include, but are not limited to, surfactants; pH control agents, such as buffers, organic acids and organic acid salts and organic and inorganic bases; glycerides; partial glycerides; glyceride derivatives; polyoxyethylene and polyoxypropylene ethers and their copolymers; sorbitan esters; polyoxyethylene sorbitan esters; carbonate salts; alkyl sulfonates; and cyclodextrins.

The solid dispersion can optionally include a number of additives and excipients that promote its stability, tableting or processing of the dispersion or suspension. Such additives and excipients include, but are not limited to, at least one coating tableting aids, water-soluble polymers, surfactants, pH modifiers, fillers, binders, pigments, disintegrants, antioxidants, lubricants, flow aids and flavorants. Examples of such components include, but are not limited to, microcrystalline cellulose; metallic salts of acids such as aluminum stearate, calcium stearate, magnesium stearate, sodium stearate, and zinc stearate; fatty acids, hydrocarbons and fatty alcohols such as stearic acid, palmitic acid, liquid paraffin, stearyl alcohol, and palmitol; fatty acid esters such as glyceryl (mono- and di-) stearates, triglycerides, glyceryl (palmitic stearic) ester, sorbitan monostearate, saccharose monostearate, saccharose monopalmitate, and sodium stearyl fumarate; alkyl sulfates such as sodium lauryl sulfate and magnesium lauryl sulfate; polymers such as polyethylene glycols, polyoxethylene glycols, and polytetrafluoroethylene; and inorganic materials such as talc and dicalcium phosphate and silicon dioxide; sugars such as lactose and xylitol; and sodium starch glycolate.

As mentioned previously herein, the pharmaceutical compositions of the present invention, upon contact with an aqueous medium, form a suspension that comprises particles which contain 2-[4-(4-chlorobenzoyl)phenoxy]-2-methyl-propanoic acid, 1-methylethyl ester. The inventors of the present invention have developed a method for determining or screening whether solid dispersions which comprise primarily amorphous 2-[4-(4-chlorobenzoyl)phenoxy]-2-methyl-propanoic acid, 1-methylethyl ester, at least one pharmaceutically acceptable polymer and optionally, at least one pharmaceutically acceptable surfactant, will form a suspension that upon contact with an aqueous medium, comprise particles that contain 2-[4-(4-chlorobenzoyl)phenoxy]-2-methyl-propanoic acid, 1-methylethyl ester.

Manufacturing Methods

The first step in manufacturing the compositions of the present invention involves preparing a solid dispersion. As discussed previously herein, methods for making solid dispersions are well known to those skilled in the art and include, but are not limited to, melt extrusion, evaporation, curing, microwaves, milling, ultra sound, spinning disc, etc. As also discussed previously herein, the solid dispersion will contain primarily amorphous 2-[4-(4-chlorobenzoyl)phenoxy]-2-methyl-propanoic acid, 1-methylethyl ester, at least one pharmaceutically acceptable polymer and, optionally, at least one pharmaceutically acceptable surfactant.

Screening Methods

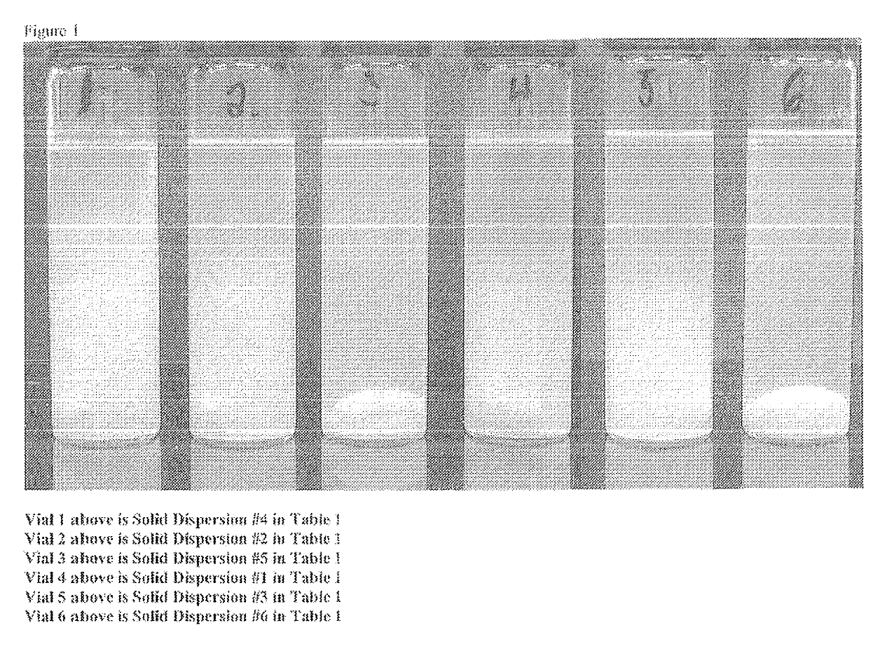

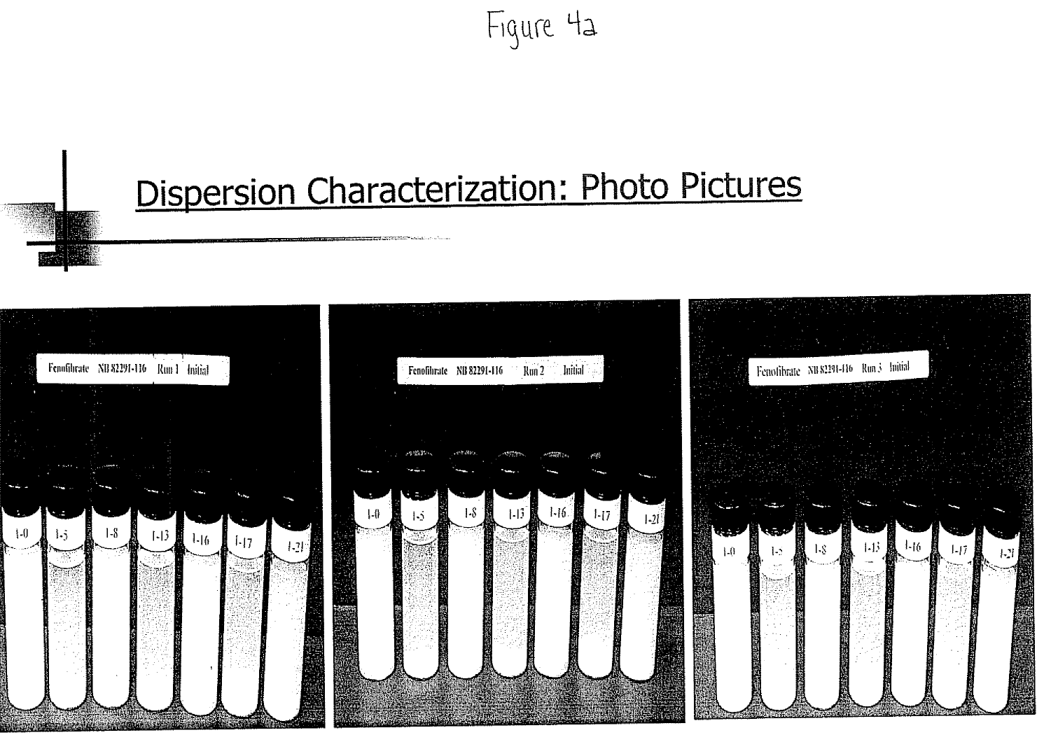

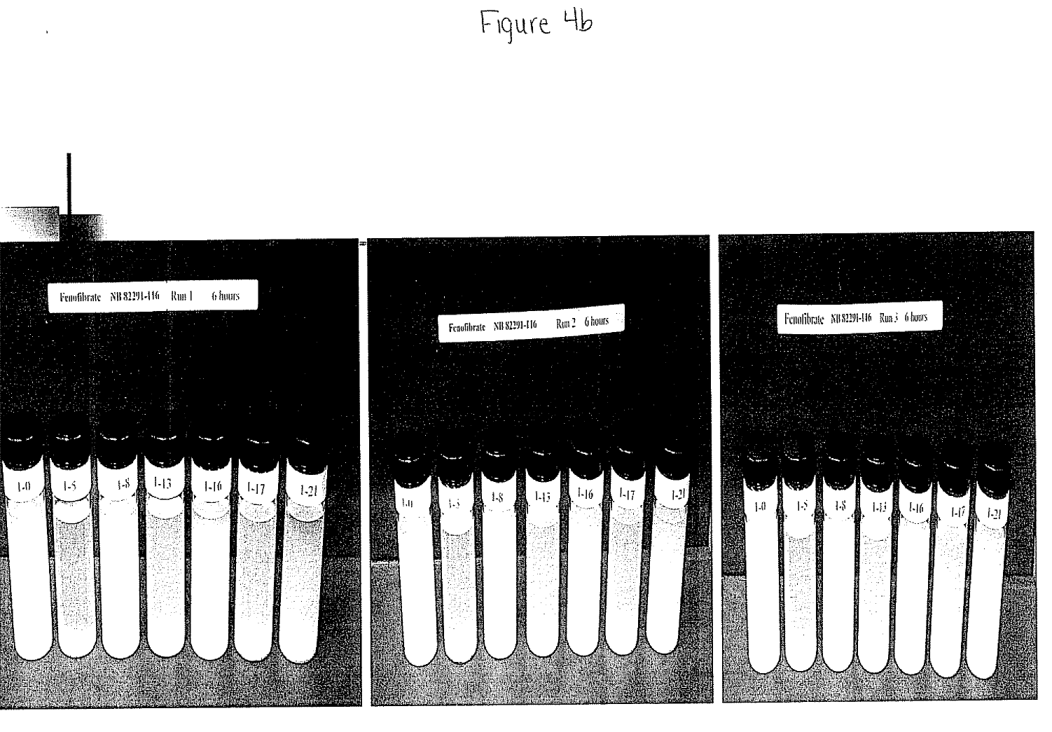

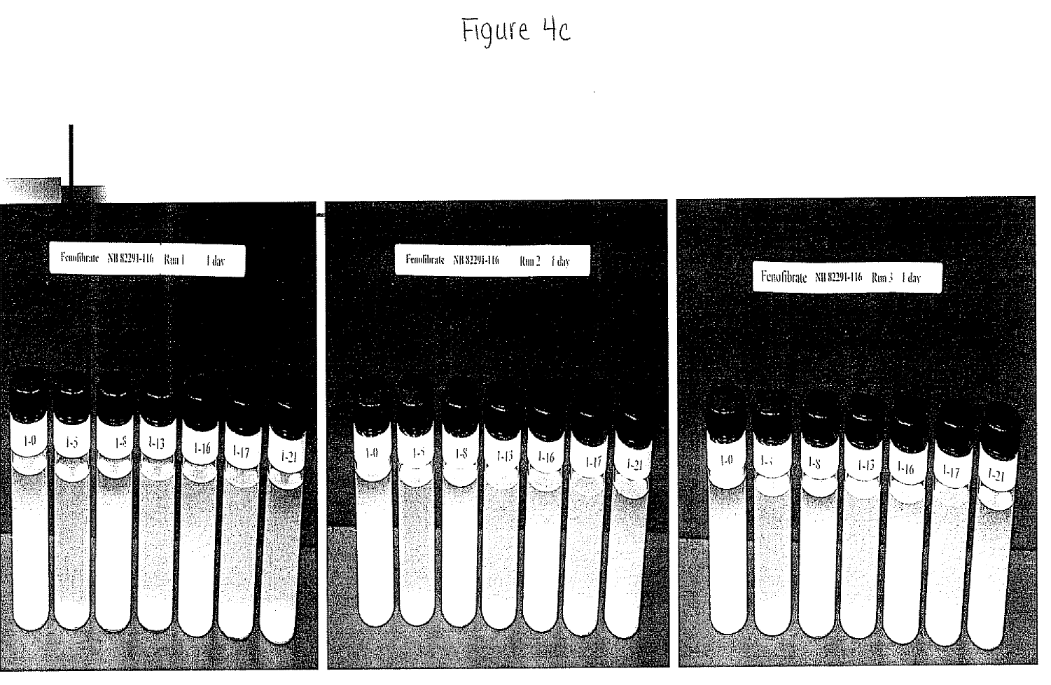

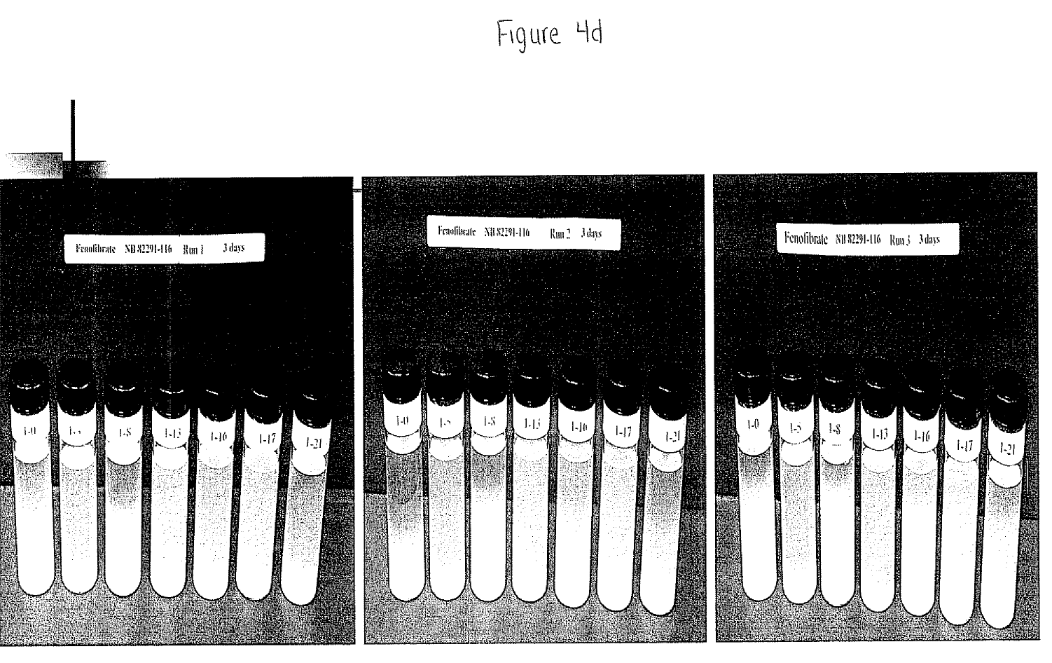

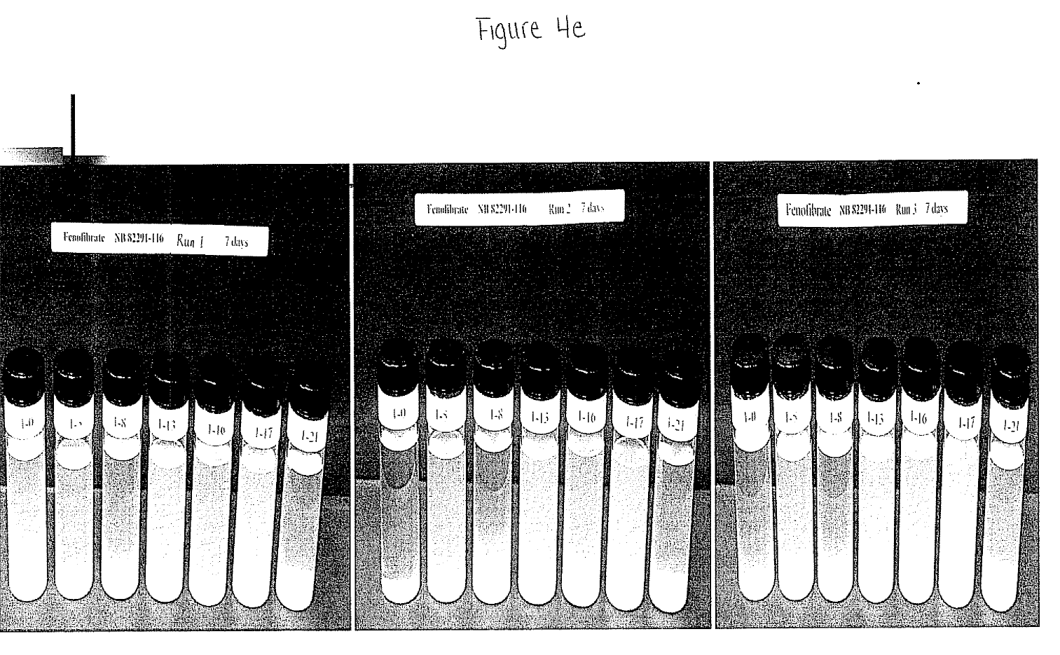

Once the solid dispersion has been formed, it is placed in an aqueous medium to form a suspension containing particles of 2-[4-(4-chlorobenzoyl)phenoxy]-2-methyl-propanoic acid, 1-methylethyl ester. Any aqueous medium can be used. Preferably, the aqueous medium is water. The pH of the aqueous medium can be adjusted if necessary by adding salts to the water. Once the solid dispersion is placed in the aqueous medium, it can be stirred until the dispersion has fully dispersed in the aqueous medium and the suspension has formed. When the solid dispersion is fully dispersed in the aqueous medium, the suspension will have a cloudy, almost milky appearance (See FIGS. 1 and 4) in the aqueous medium. After the solid dispersion is fully dispersed in the aqueous medium and the suspension has been formed, any stirring can be stopped and the fully dispersed suspension is left at room temperature for a period of from about fifteen (15) minutes to about seven (7) days, preferably for a period of about thirty (30) minutes to about five (5) days, more preferably from about one (1) hour to about two (2) days. The period of time in which the suspension is left at room temperature is not critical. If the suspension still has a cloudy, almost milky appearance, this indicates that when the solid dispersion containing the at least one pharmaceutically acceptable polymer and the primarily amorphous 2-[4-(4-chlorobenzoyl)phenoxy]-2-methyl-propanoic acid, 1-methylethyl ester, and optionally, the at least one pharmaceutically acceptable surfactant is contacted with an aqueous medium, it forms a suspension comprising particles containing 2-[4-(4-chlorobenzoyl)phenoxy]-2-methyl-propanoic acid, 1-methylethyl ester. The particles contained within such a suspension typically have a particle size of from about 100 nm to about 10,000 nm, preferably from about 200 nm to about 5000 nm. As mentioned herein, the suspension formed as described herein is stable. As used herein, the term “stable” refers to the fact that the suspension does not coagulate and does not form an agglomerate.

In contrast, if the suspension no longer has a cloudy, almost milky appearance, but has instead coagulated and possibly even formed an agglomerate (See FIGS. 1 and 4), this indicates that the at least one pharmaceutically acceptable polymer, the at least one pharmaceutically acceptable surfactant (if present) or the at least one pharmaceutically acceptable polymer and at least one pharmaceutically acceptable surfactant is not suitable, and that solid dispersions formed containing said at least one pharmaceutically acceptable polymer, the at least one pharmaceutically acceptable surfactant (if present) or the at least one pharmaceutically acceptable polymer and at least one pharmaceutically acceptable surfactant will not, when contacted with an aqueous medium, form a suspension comprising particles containing 2-[4-(4-chlorobenzoyl)phenoxy]-2-methyl-propanoic acid, 1-methylethyl ester.

Bioavailability

The inventors of the present invention have found that a suspension comprising particles containing 2-[4-(4-chlorobenzoyl)phenoxy]-2-methyl-propanoic acid, 1-methylethyl ester have improved bioavailability compared to the reference formulation.

The inventors of the present invention have also discovered that when the pharmaceutical compositions of the present invention contain as an active agent, primarily amorphous 2-[4-(4-chlorobenzoyl)phenoxy]-2-methyl-propanoic acid, 1-methylethyl ester, that said compositions lack a significant food effect on oral administration to subjects when compared to the reference formulation.

As used herein, the term “lacks a significant food effect” means that a composition of the present invention containing primarily amorphous 2-[4-(4-chlorobenzoyl)phenoxy]-2-methyl-propanoic acid, 1-methylethyl ester, when administered in the fed state to a subject is bioequivalent to the same composition when administered in the fasted state to a subject. Two products or methods are bioequivalent if the 90% confidence intervals (CI) for the individual ratios of fed AUC to fasted AUC and fed Cₘₐₓ to fasted Cₘₐₓ are between 0.70 to 1.43, preferably between 0.80 to 1.25.

Specifically, pharmacokinetic studies in fed and fasted subjects were conducted using a composition of the present invention containing primarily amorphous 2-[4-(4-chlorobenzoyl)phenoxy]-2-methyl-propanoic acid, 1-methylethyl ester as the active agent. It was unexpectedly discovered that these compositions lacked significant food effect on oral administration. In contrast, in a separate study, compositions containing 200 mg of conventional microcrystalline 2-[4-(4-chlorobenzoyl)phenoxy]-2-methyl-propanoic acid, 1-methylethyl ester exhibited significant food effect on oral administration.

Because the oral compositions of the present invention lack a significant food effect on oral administration a number of benefits are realized. For example, subject convenience is increased which may lead to increasing subject compliance since the subject does not need to ensure that they are taking a dose either with or without food. This is significant, because when there is poor subject compliance, an exacerbation of the medical condition for which the drug is being prescribed may be observed. For example, disease symptoms associated with suboptimal control of blood lipids may occur when there is poor subject compliance with 2-[4-(4-chlorobenzoyl)phenoxy]-2-methyl-propanoic acid, 1-methylethyl ester.

Methods of Treating

The present invention also provides a method of treating a subject suffering from dyslipidemia and/or dyslipoproteinemia. The method comprises the step of orally administering a therapeutically effective amount of a pharmaceutical composition of the present invention to a subject in need thereof. The subject can be a mammal, such as a human being, that is suffering from dyslipidemia and/or dyslipoproteinemia.

The present invention will be understood more clearly from the following non-limiting representative examples.

### EXAMPLE 1

### Preparation of Solid Dispersion Via Melt Extrusion

Seven (7) different solid dispersion formulations comprising primarily amorphous 2-[4-(4-chlorobenzoyl)phenoxy]-2-methyl-propanoic acid, 1-methylethyl ester and at least one pharmaceutically acceptable polymer were prepared. In some of the solid dispersion formulations at least one pharmaceutically acceptable surfactant was included. The composition of each of these solid dispersions is shown in Table 1 below. Solid dispersion #7 is a control. Each of the solid dispersions having the composition shown in Table 1 below form a colloidal dispersion upon contact with an aqueous medium, such as water.

TABLE 1

| | Solid Dispersion | | | | | | |
|---|---|---|---|---|---|---|---|
| | | | | | | | #7 |
| | #1 | #2 | #3 | #4 | #5 | #6 | (Control) |
| surface active | None | Labrafil ® | Tween ® | Cremophor ® | Labrafil ® | Span ® | None |
| agent(s) type | | M1944 | 85 | RH-40 + Miglyol ® 812 N | M1944 | 20 | |
| | | CS¹ | | | CS | | |
| Surface active | n.a. | 2 | 2 | 2.5/2.5 | 5 | 5 | n.a. |
| agent [%] by | | | | | | | |
| weight | | | | | | | |
| Active Agent | 15 | 15 | 15 | 15 | 15 | 15 | 15 |
| [%] by | | | | | | | |
| weight² | | | | | | | |
| Copovidone ® | 84 | 82 | 82 | 60 | 60 | 79 | |
| VA 64 | | | | | | | |
| (BASF) | | | | | | | |
| PVP | | | | 19 | 19 | | |
| (Kollidon ® | | | | | | | |
| 25, BASF) | | | | | | | |
| HPMCP 55S | | | | | | | 84 |
| Aerosil ® 200 | 1 | 1 | 1 | 1 | 1 | 1 | 1 |

¹Labrafil ® M 1944 CS is glyceryl and polyethylene glycol ester that is available from Gattefosse
²2-[4-(4-chlorobenzoyl)phenoxy]-2-methyl-propanoic acid, 1-methylethyl ester.

Solid dispersion #1 having the composition shown above in Table 1 was prepared as follows. Specifically, crystalline 2-[4-(4-chlorobenzoyl)phenoxy]-2-methyl-propanoic acid, 1-methylethyl ester (Helm, Germany) was blended with Copovidon® and Aerosil® 200. This powder mixture was fed by a loss in weight feeder system into a twin-screw extruder having 18 mm screw diameter. Extrusion was performed at a temperature of about 120° C. resulting in a viscous melt leaving the extruder nozzle. The active agent containing melt was directly formed into tablets by calendaring between two counter-rotating rollers having depressions on the surface of the rollers according to the tablet dimension. The calendared tablets were cooled to room temperature on a conveyor belt. Tablet weight was about 360 mg corresponding to 54 mg of 2-[4-(4-chlorobenzoyl)phenoxy]-2-methyl-propanoic acid, 1-methylethyl ester per tablet.

Solid dispersions #2-#6 having the composition shown above in Table 1 were prepared as follows. Each of these solid dispersions (#2-#6) were prepared in the same manner as solid dispersion #1, described above, except that the liquid excipients were granulated with the polymer(s). These polymer/excipient granules were blended with crystalline 2-[4-(4-chlorobenzoyl)phenoxy]-2-methyl-propanoic acid, 1-methylethyl ester (Helm, Germany) prior to extrusion. Extrusion was performed at a temperature of about 120° C. as with solid dispersion

Solid dispersion #7 having the composition shown above in Table 1 was prepared as follows. Solid dispersion #7 was prepared in the same manner as solid dispersion #1, described above, however, extrusion was performed at a temperature of about 165° C.

Each of solid dispersions #1-#7 were examined by differential scanning calorimetry (DSC) at 110° C. per minute, the technique for which is well known in the art. In all of the samples, no crystalline 2-[4-(4-chlorobenzoyl)phenoxy]-2-methyl-propanoic acid, 1-methylethyl ester was detected.

### EXAMPLE 2

### Formation of Suspensions from the Solid Dispersions of Example 1

Melt extruded solid dispersions #1-#6 prepared as described above in Example 1 were dispersed in an aqueous medium, specifically, water, to form a suspension. FIG. 1 shows each of the suspensions formed by each of solid dispersions #1-#6 after dispersion in the water. The particle size was measured by laser diffraction techniques, the techniques of which are well known to those skilled in the art. Table 2, below, shows the particle size of each of the nanosuspensiosn. The measurements of the size of the particles in each of the suspensions are reported as D50 (μm) and D90 (μm).

TABLE 2

| Dispersion # | D50 (μm) | D90 (μm) |
|---|---|---|
| #1 | 3.43 | 5.87 |
| #2 | 5.47 | 42.14 |
| #3 | 4.34 | 10.73 |
| #4 | 2.62 | 4.37 |
| #5 | 37.4 | 68.77 |
| #6 | 30.91 | 56.86 |

### EXAMPLE 3

### Comparison of the Bioavailability of Primarily Amorphous versus Crystalline 2-[4-(4-chlorobenzoyl)phenoxy]-2-methyl-propanoic acid, 1-methylethyl ester

2-[4-(4-chlorobenzoyl)phenoxy]-2-methyl-propanoic acid, 1-methylethyl ester is present in an amorphous form in the melt-extruded solid dispersions shown above in solid dispersion #1-#6 in Example 1. However, the amorphous 2-[4-(4-chlorobenzoyl)phenoxy]-2-methyl-propanoic acid, 1-methylethyl ester contained with each of these solid dispersions was found to convert to crystalline form on storage or on exposure to moisture. In view of this, a dog model was used to evaluate the effect of crystalline 2-[4-(4-chlorobenzoyl)phenoxy]-2-methyl-propanoic acid, 1-methylethyl ester on bioavailability using solid dispersion #2 described above in Example 1.

More specifically, the melt extrudate of solid dispersion #2 was manufactured as discussed in Example 1 above. In addition, the 2-[4-(4-chlorobenzoyl)phenoxy]-2-methyl-propanoic acid, 1-methylethyl ester containing extrudate was milled and the milled extrudate was blended with 1.06% Aerosil® 200 and 1.3% sodium stearyl fumarate. This blended mixture was compressed into tablets. The final tablets were film-coated by using a ready-to-use excipient mixture (Opadry®, Colorcon) in a drum coater (film-coating of an aqueous dispersion of Opadry®). The amount of film-coating on the tablet surface was 2.3% relative to the total weight of the tablet. The resulting film coated tablets contained 160 mg of amorphous 2-[4-(4-chlorobenzoyl)phenoxy]-2-methyl-propanoic acid, 1-methylethyl ester. The composition of this tablet is shown in Table 3 below. An aliquot of these tablets were allocated for use as a “Control” formulation as described in more detail below.

TABLE 3

| | Tablet Formulation |
|---|---|
| Component | (%, by weight) |
| 2-[4-(4-chlorobenzoyl)phenoxy]-2-methyl-propanoic | 14.64 |
| acid, 1-methylethyl ester | |
| Copovidone ® VA64 | 80.03 |
| Labrafil ® | 1.95 |
| Aerosil ® | 2.08 |
| Sodium Stearyl Fumarate | 1.30 |
| Film Coating (Opadry ®) | 2.3 |

The remaining tablets were crystallized to different extents by exposing the tablets for 52 days to approximately 75% relative humidity in an open dish for different periods of time. More specifically, the tablets were stored at room temperature in an open dish in a chamber maintained at constant relative humidity of approximately 75% by a saturated salt solution. The amount of crystalline active agent was measured on the dried and ground tablet samples by differential scanning calorimetry. In 21 days, only a small fraction of the active agent was expected to crystallize whereas in 52 days equilibrium was attained and the active agent had crystallized to the limit of solubility.

For administration to the dogs, a reference composition (TRICOR® 67 mg capsule) was compared to the Control and the stressed tablets prepared as described above. The 160 mg Control and stressed tablets were cut using a knife to provide an approximately 54 mg dose by weight. A single dose of approximately 54 mg (either the Control or one of the stressed tablets) was provided to fasted dogs. The plasma samples were analyzed using HPLC-MS/MS, which is a standard technique known to those skilled in the art.

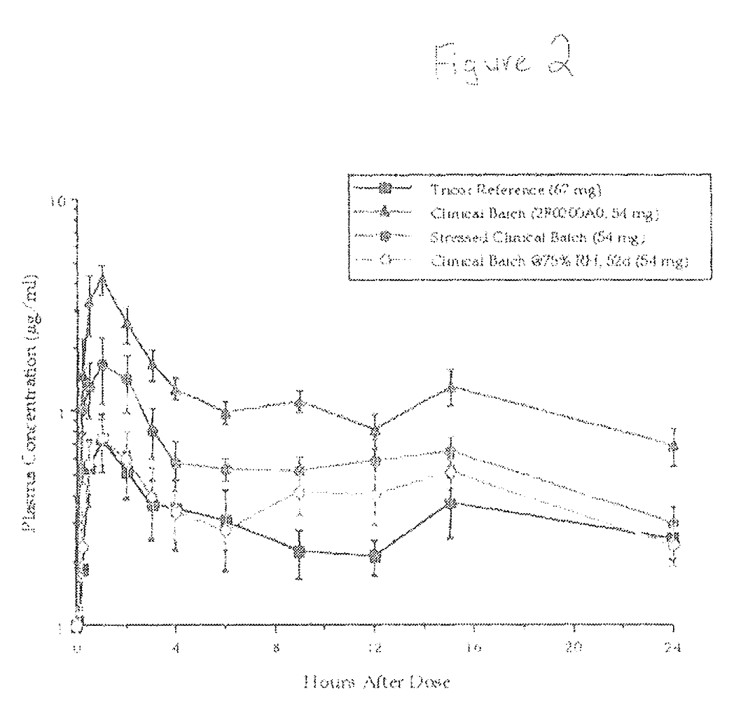

FIG. 2 shows the mean plasma concentration of 2-[4-(4-chlorobenzoyl)phenoxy]-2-methyl-propanoic acid after an approximately 54 mg single oral dose of 2-[4-(4-chlorobenzoyl)phenoxy]-2-methyl-propanoic acid, 1-methylethyl ester in fasted dogs. The concentration decreases markedly for the samples containing 12% crystalline drug as compared to no crystalline drug in the Control (or clinical batch) tablets.

Table 4 below, shows the pharmacokinetic parameters for the dog studies. The point estimate for AUC decreases as the crystallized drug increases. This indicates that the crystalline drug adversely affects the bioavailability of the formulation. Qualitatively, the study shows the importance of maintaining the 2-[4-(4-chlorobenzoyl)phenoxy]-2-methyl-propanoic acid, 1-methylethyl ester in a primarily amorphous form.

TABLE 4

| | | | AUC₀₋₂₄ | AUC Pt. |
|---|---|---|---|---|
| Composition | Cₘₐₓ (μg/ml) | Tₘₐₓ (hr) | (μg · hr/ml) | Estimate |
| TRICOR ®^{a} | 0.83 ± 0.58 | 1.4 ± 0.9 | 7.6 ± 4.63 | |
| Control^{b} | 5.34 ± 3.01 | 1.2 ± 0.8 | 30.08 ± 12.07 | Ref. |
| Stressed at 75% | 2.55 ± 1.35 | 1.0 ± 0.5 | 15.08 ± 6.86 | 0.41 |
| RH for 12 | | | | |
| days^{b} | | | | |
| Stressed at 75% | 0.94 ± 0.41 | 0.9 ± 0.6 | 9.7 ± 4.02 | 0.37 |
| RH for 52 | | | | |
| days^{b} | | | | |

^{a}reference formulation, 67 mg capsule.
^{b}approximately 54 mg solid dose containing 2-[4-(4-chlorobenzoyl)phenoxy]-2-methyl-propanoic acid, 1-methylethyl ester prepared as described herein.

The study shows that bioavailability in dogs is adversely affected by the crystallization of 2-[4-(4-chlorobenzoyl)phenoxy]-2-methyl-propanoic acid, 1-methylethyl ester in the melt extruded solid dispersion. The bioavailability decreased with an increase in the degree of crystallinity. The study indicates the importance of maintaining the drug in the primarily amorphous form in the 2-[4-(4-chlorobenzoyl)phenoxy]-2-methyl-propanoic acid, 1-methylethyl ester melt extruded solid dispersion.

### EXAMPLE 4

### Study of the Effect of Food on the Bioavailability of Fenofibrate

The purpose of this study was to determine the effect of food on the bioavailability of fenofibrate from the 160 mg tablet “Control” formulation described in Example 3, which will also be referred to herein in this Example as the “test formulation” and the 200 mg capsule reference formulation described previously herein (which shall be referred to in this Example as the “reference formulation”). This study was a Phase 1, single-dose, open-label study that was conducted according to a four-period, randomized crossover design. Twenty (20) subjects entered the study and were to receive one of four sequences of Regimen A (one tablet of the test formulation under fasting conditions), Regimen B (one tablet of the test formulation following a low-fat meal), Regimen C (one tablet of the test formulation following a high-fat meal) and Regimen D (one capsule of the reference formulation following a low-fat meal) in the morning on Study Day 1 of each period. The sequences of regimens were such that each subject received all four regimens upon completion of the study. The washout period for the study was 14 days. Adult male and female subjects in general good health were selected to participate in the study. Eighteen (18) of the twenty (20) subjects that entered the study completed the study. For the 18 subjects included in the pharmacokinetic analyses, the mean age was 31.8 years (ranging from 20 to 45 years), the mean weight was 73.6 kg (ranging from 56.0 to 89.0 kg) and the mean height was 175.4 cm (ranging from 159.0 to 193.0 cm).

Subjects were confined to the study site and supervised for approximately 6 days in each study period. Confinement in each period began in the afternoon on Study Day −1 (1 day prior to the dosing day) and ended after the collection of the 120-hour blood samples and scheduled study procedures were completed on the morning of Study Day 6. Strenuous activity during the confinement was not permitted.

With the exception of the breakfast on Study Day 1 in each period, subjects received a standard diet, providing approximately 34% calories from fat per day, for all meals during confinement. For those subjects assigned to Regimen A, no food or beverage, except for water to quench thirst, was allowed beginning 10 hours before dosing and continuing until after the collection of the 4-hour blood sample on the following day (Study Day 1). No fluids were allowed for 1 hour before dosing and 1 hour after dosing. On Study Day 1, those subjects assigned to Regimens B and D received a low-fat breakfast that provided approximately 520 Kcal and 30% of calories from fat beginning 30 minutes prior to dosing. On Study Day 1, those subjects assigned to Regimen C received a high-fat breakfast that provided approximately 1000 Kcal and 50% of calories from fat beginning 30 minutes prior to dosing.

On Study Day 1, all subjects were served lunch following collection of the 4-hour blood sample, dinner following collection of the 10-hour blood sample, and a snack approximately 4 hours after dinner. The meal content with the exception of breakfast was identical on the intensive pharmacokinetic sampling days (Study Day 1) of all four periods.

Blood samples were collected from the subjects by venipuncture into 5 mL evacuated collection tubes containing potassium oxalate plus sodium fluoride prior to dosing (0 hours) and at 1, 2, 3, 4, 5, 6, 7, 8, 9, 10, 11, 12, 18, 24, 48, 72, 96 and 120 hours after dosing (Study Day 1) in each period. The blood samples were centrifuged to separate the plasma. The plasma samples were stored frozen until analyzed.

Plasma concentrations of fenofibric acid were determined using a validated liquid chromatographic method with mass spectrometric detection.

Values for the pharmacokinetic parameters of fenofibric acid were estimated using noncompartmental methods. First, the maximum observed plasma concentration (Cₘₐₓ) and the time to Cₘₐₓ (peak time, Tₘₐₓ) were determined directly from the plasma concentration-time data. Second, the value of the terminal phase elimination rate constant (λ_{z}) was obtained from the slope of the least squares linear regression of the logarithms of the plasma concentration versus time data from the terminal log-linear phase of the profile. A minimum of three concentration-time data points was used to determine λ_{z}. The terminal phase elimination half-life (t_{1/2}) was calculated as ln(2)/λ_{z}.

Third, the area under the plasma concentration-time curve (AUC) from time 0 to time of the last measurable concentration (AUCₜ) was calculated by the linear trapezoidal rule. The AUC was extrapolated to infinite time by dividing the last measurable plasma concentration (Cₜ) by λ_{z} to give AUC from time 0 to infinite time (AUC_{∞}).

An analysis of variance (ANOVA) was performed for Tₘₐₓ and the natural logarithms of Cₘₐₓ and AUC. The model included effects for sequence, subject nested within sequence, period and regimen. The effects of sequence, period and regimen were fixed, while the effect of subject was random. For the test on sequence effects, the denominator sum of squares for the F statistic was the sum of squares for subject nested within sequence. For the tests on period and regimen effects, the denominator sum of squares was the residual sum of squares. The statistical tests were performed at a significance level of 0.05.

The bioavailability of the high-fat meal regimen (Regimen C) relative to that of the fasting regimen (Regimen A) was assessed by the two one-sided tests procedure via 90% confidence intervals. Absence of food effect was concluded if the 90% confidence intervals from the analyses of the natural logarithms of AUC and Cₘₐₓ were within the 0.80 to 1.25 range. The bioavailability of the low-fat meal test regimen (Regimen B) relative to that of the low-fat meal reference regimen (Regimen D) was assessed by the two one-sided tests procedure via 90% confidence intervals. Bioequivalence was concluded if the 90% confidence intervals from the analyses of the natural logarithms of AUC and Cₘₐₓ were within the 0.80 to 1.25 range.

Mean±standard deviation (SD) pharmacokinetic parameters of fenofibric acid after administration of the four regimens are listed below in Table 5.

TABLE 5

| | Regimen A | Regimen B | Regimen C | Regimen D |
|---|---|---|---|---|
| | Test | Test | Test | Reference |
| Pharmacokinetic | Formulation, | Formulation, | Formulation, | Formulation, |
| Parameters | Fasting | Low-Fat Meal | High-Fat Meal | Low-Fat Meal |
| (Units) | (N = 18) | (N = 18) | (N = 18) | (N = 18) |
| Tₘₐₓ (h) | 1.8 ± 0.8 | 3.9 ± 1.1 | 3.7 ± 1.4* | 4.6 ± 0.9 |
| Cₘₐₓ (μg/mL) | 8.13 ± 3.00 | 7.30 ± 1.97 | 8.50 ± 2.13 | 7.27 ± 2.74 |
| AUCₜ (μg · h/mL) | 116.4 ± 60.9 | 115.8 ± 58.8† | 127.0 ± 61.2* | 133.7 ± 76.1 |
| AUC_{∞} (μg · h/mL) | 119.8 ± 65.7 | 118.2 ± 61.9† | 129.5 ± 64.1* | 138.4 ± 81.6 |
| t_{1/2}$ (h) | 16.8 | 15.8† | 15.7* | 17.6 |

*Statistically significantly different from Regimen A (ANOVA, p < 0.05).
†Statistically significantly different from Regimen D (ANOVA, p < 0.05).
$Harmonic mean; evaluations t_{1/2} were based on statistical tests for λ_{z}.

The relative bioavailability and food effect results are shown below in Tables 6 and 7, respectively.

TABLE 6
Relative Bioavailability

| | | Central Values* | | Relative Bioavailability | |
|---|---|---|---|---|---|
| Test vs. | Pharmacokinetic | Test | Reference | Point | 90% Confidence |
| Reference | Parameter | Formulation | Formulation | Estimate⁺ | Interval |
| B vs. D | Cₘₐₓ | 7.1 | 6.9 | 1.031 | 0.891-1.193 |
| B vs. D | AUCₜ | 107.1 | 119.5 | 0.896 | 0.829-0.968 |
| B vs. D | AUC_{∞} | 108.8 | 122.7 | 0.887 | 0.821-0.959 |

*Antilogarithm of the least squares means for logarithms.
⁺Antilogarithm of the difference (test formulation minus reference formulation) of the least squares means for logarithms.

TABLE 7
Food Effect

| | | Central Values* | | Relative Bioavailability | |
|---|---|---|---|---|---|
| Test vs. | Pharmacokinetic | Test | Reference | Point | 90% Confidence |
| Reference | Parameter | Formulation | Formulation | Estimate⁺ | Interval |
| C vs. A | Cₘₐₓ | 8.2 | 7.7 | 1.074 | 0.928-1.243 |
| C vs. A | AUCₜ | 117.7 | 106.2 | 1.108 | 1.026-1.198 |
| C vs. A | AUC_{∞} | 119.7 | 108.5 | 1.104 | 1.021-1.193 |

*Antilogarithm of the least squares means for logarithms.
⁺Antilogarithm of the difference (test formulation minus reference formulation) of the least squares means for logarithms.

The test formulation (Regimen B) was bioequivalent to the reference formulation (Regimen D) because the 90% confidence intervals for evaluating bioequivalence were within the 0.80 to 1.25 range. In addition, statistical proof of the lack of food effect on the test formulation was provided by the 90% confidence intervals for evaluating food effect (Regimen C versus Regimen A) which were within the 0.80 to 1.25 range.

The regimens tested were generally well tolerated by the subjects. No clinically significant physical examination results, or vital signs or laboratory measurements were observed during the course of the study. No differences were seen among the regimens with respect to adverse event profiles. There were no apparent differences among the regimens with regard to safety.

### EXAMPLE 5

### Study of the Effect of Food on the Bioavailability of Fenofibrate

The purpose of this study was to determine the effect of food on the bioavailability of fenofibrate from a 54 mg tablet formulation made as described in Example 3, which will also be referred to herein in this Example as the “test formulation” and a 67 mg capsule reference formulation described previously herein (which shall be referred to in this Example as the “reference formulation”). This study was a Phase 1, single-dose, open-label study that was conducted according to a four-period, randomized crossover design. Twenty (20) subjects entered the study and were to receive one of four sequences of Regimen A (one tablet of the test formulation under fasting conditions), Regimen B (one tablet of the test formulation following a low-fat meal), Regimen C (one tablet of the test formulation following a high-fat meal) and Regimen D (one capsule of the reference formulation following a low-fat meal) in the morning on Study Day 1 of each period. The sequences of regimens were such that each subject received all four regimens upon completion of the study. The washout period for the study was 14 days. Adult male and female subjects in general good health were selected to participate in the study. For the twenty (20) subjects who participated in this study, the mean age was 33.5 years (ranging from 24 to 44 years), the mean weight was 75.4 kg (ranging from 56.0 to 104.0 kg) and the mean height was 174.3 cm (ranging from 155.0 to 189.0 cm). All of the twenty (20) subjects that entered the study completed the study.

Subjects were confined to the study site and supervised for approximately 6 days in each study period. Confinement in each period began in the afternoon on Study Day 1 (1 day prior to the dosing day) and ended after the collection of the 120-hour blood samples and scheduled study procedures were completed on the morning of Study Day 6. Strenuous activity during the confinement was not permitted.

With the exception of the breakfast on Study Day 1 in each period, subjects received a standard diet, providing approximately 34% calories from fat per day, for all meals during confinement. For those subjects assigned to Regimen A, no food or beverage, except for water to quench thirst, was allowed beginning 10 hours before dosing and continuing until after the collection of the 4-hour blood sample on the following day (Study Day 1). No fluids were allowed for 1 hour before dosing and 1 hour after dosing. On Study Day 1, those subjects assigned to Regimens B and D received a low-fat breakfast that provided approximately 520 Kcal and 30% of calories from fat beginning 30 minutes prior to dosing. On Study Day 1, those subjects assigned to Regimen C received a high-fat breakfast that provided approximately 1000 Kcal and 50% of calories from fat beginning 30 minutes prior to dosing.

On Study Day 1, all subjects were served lunch following collection of the 4-hour blood sample, dinner following collection of the 10-hour blood sample and a snack approximately 4 hours after dinner. The meal content with the exception of breakfast was identical on the intensive pharmacokinetic sampling days (Study Day 1) of all four periods.

Blood samples of the subjects were collected from the subjects by venipuncture into 5 mL evacuated collection tubes containing potassium oxalate plus sodium fluoride prior to dosing (0 hours) and at 1, 2, 3, 4, 5, 6, 7, 8, 9, 10, 11, 12, 18, 24, 48, 72, 96 and 120 hours after dosing (Study Day 1) in each period. The blood samples were centrifuged to separate the plasma. The plasma samples were stored frozen until analyzed.

Plasma concentrations of fenofibric acid were determined using a validated liquid chromatographic method with mass spectrometric detection.

Values for the pharmacokinetic parameters of fenofibric acid were estimated using noncompartmental methods. First, the maximum observed plasma concentration (Cₘₐₓ) and the time to Cₘₐₓ (peak time, Tₘₐₓ) were determined directly from the plasma concentration-time data. Second, the value of the terminal phase elimination rate constant (λ_{z}) was obtained from the slope of the least squares linear regression of the logarithms of the plasma concentration versus time data from the terminal log-linear phase of the profile. A minimum of three concentration-time data points was used to determine λ_{z}. The terminal phase elimination half-life (t_{1/2}) was calculated as ln(2)/λ_{z}.

Third, the area under the plasma concentration-time curve (AUC) from time 0 to time of the last measurable concentration (AUCₜ) was calculated by the linear trapezoidal rule. The AUC was extrapolated to infinite time by dividing the last measurable plasma concentration (Cₜ) by λ_{z} to give AUC from time 0 to infinite time (AUC_{∞}).

An analysis of variance (ANOVA) was performed for Tₘₐₓ and the natural logarithms of Cₘₐₓ and AUC. The model included effects for sequence, subject nested within sequence, period and regimen. The effects of sequence, period and regimen were fixed, while the effect of subject was random. For the test on sequence effects, the denominator sum of squares for the F statistic was the sum of squares for subject nested within sequence. For the tests on period and regimen effects, the denominator sum of squares was the residual sum of squares. The statistical tests were performed at a significance level of 0.05.

The bioavailability of the high-fat meal regimen (Regimen C) relative to that of the fasting regimen (Regimen A) was assessed by the two one-sided tests procedure via 90% confidence intervals. Absence of food effect was concluded if the 90% confidence intervals from the analyses of the natural logarithms of AUC and Cₘₐₓ were within the 0.80 to 1.25 range. The bioavailability of the low-fat meal test regimen (Regimen B) relative to that of the low-fat meal reference regimen (Regimen D) was assessed by the two one-sided tests procedure via 90% confidence intervals. Bioequivalence was concluded if the 90% confidence intervals from the analyses of the natural logarithms of AUC and Cₘₐₓ were within the 0.80 to 1.25 range.

Mean±standard deviation (SD) pharmacokinetic parameters of fenofibric acid after administration of the four regimens are listed in below in Table 8.

TABLE 8

| | Regimen A | Regimen B | Regimen C | Regimen D |
|---|---|---|---|---|
| | Test | Test | Test | Reference |
| Pharmacokinetic | Formulation, | Formulation, | Formulation, | Formulation, |
| Parameters | Fasting | Low-Fat Meal | High-Fat | Low-Fat Meal |
| (Units) | (N = 20) | (N = 20) | Meal (N = 20) | (N = 20) |
| Tₘₐₓ (h) | 1.9 ± 0.9 | 3.8 ± 1.9† | 4.1 ± 1.7* | 5.5 ± 2.3 |
| Cₘₐₓ (μg/mL) | 3.13 ± 1.23 | 2.70 ± 0.77 | 2.99 ± 1.04 | 2.54 ± 0.87 |
| AUCₜ (μg · h/mL) | 52.6 ± 19.7 | 50.8 ± 16.0† | 53.1 ± 19.3 | 56.7 ± 17.8 |
| AUC_{∞} (μg · h/mL) | 53.7 ± 20.4 | 51.9 ± 16.5† | 54.1 ± 19.7 | 58.6 ± 18.6 |
| t_{1/2}$ (h) | 18.0 | 17.4† | 16.9 | 20.2 |

*Statistically significantly different from Regimen A (ANOVA, p < 0.05).
†Statistically significantly different from Regimen D (ANOVA, p < 0.05).
$Harmonic mean; evaluations of t_{1/2} were based on statistical tests for λ_{z}.

The relative bioavailability and food effect results are shown below in Tables 9 and 10, respectively.

TABLE 9
Relative Bioavailability

| | | Central Values* | | Relative Bioavailability | |
|---|---|---|---|---|---|
| Test vs. | Pharmacokinetic | Test | Reference | Point | 90% Confidence |
| Reference | Parameter | Formulation | Formulation | Estimate⁺ | Interval |
| B vs. D | Cₘₐₓ | 2.6 | 2.4 | 1.074 | 0.945-1.220 |
| B vs. D | AUCₜ | 48.6 | 54.4 | 0.894 | 0.842-0.950 |
| B vs. D | AUC_{∞} | 49.6 | 56.2 | 0.883 | 0.832-0.938 |

*Antilogarithm of the least squares means for logarithms.
⁺Antilogarithm of the difference (test formulation minus reference formulation) of the least squares means for logarithms.

TABLE 10
Food Effect

| | | Central Values* | | Relative Bioavailability | |
|---|---|---|---|---|---|
| Test vs. | Pharmacokinetic | Test | Reference | Point | 90% Confidence |
| Reference | Parameter | Formulation | Formulation | Estimate⁺ | Interval |
| C vs. A | Cₘₐₓ | 2.9 | 2.9 | 0.983 | 0.865-1.117 |
| C vs. A | AUCₜ | 50.0 | 49.6 | 1.007 | 0.948-1.071 |
| C vs. A | AUC_{∞} | 50.9 | 50.6 | 1.006 | 0.948-1.068 |

*Antilogarithm of the least squares means for logarithms.
+Antilogarithm of the difference (test formulation minus reference formulation) of the least squares means for logarithms.

The test formulation (Regimen B) was bioequivalent to the reference formulation (Regimen D) because the 90% confidence intervals for evaluating bioequivalence were within the 0.80 to 1.25 range. In addition, statistical proof of the lack of food effect on the test formulation was provided by the 90% confidence intervals for evaluating food effect (Regimen C versus Regimen A) which were within the 0.80 to 1.25 range.

### EXAMPLE 6

### Additional Preparation of Solid Dispersions Via Melt Extrusion

Seven (7) different solid dispersion formulations comprising primarily amorphous 2-[4-(4-chlorobenzoyl)phenoxy]-2-methyl-propanoic acid, 1-methylethyl ester and at least one pharmaceutically acceptable polymer were prepared. In some of the solid dispersion formulations at least one pharmaceutically acceptable surfactant was included. The composition of each of these solid dispersions is shown in Table 11 below. Each of the solid dispersions having the composition shown in Table 11 below form a colloidal dispersion upon contact with an aqueous medium, such as water.

TABLE 11

| | Solid Dispersion | | | | | | |
|---|---|---|---|---|---|---|---|
| | #1-0 | #1-5 | #1-8 | 1-13 | #1-16 | #1-17 | #1-21 |
| surface active | None | Labrafil ® | Tween ® | Lauroglycol | Cremophor ® | Labrafil ® | Span ® |
| agent(s) type | | M1944 | 85 | FCC¹ | RH-40 + Miglyol ® 812 N | M1944 | 20 |
| | | CS¹ | | | | CS² | |
| Surface active | n.a. | 2 | 2 | 5 | 2.5/2.5 | 5 | 5 |
| agent [%] by | | | | | | | |
| weight | | | | | | | |
| Active Agent | 15 | 15 | 15 | 15 | 15 | 15 | 15 |
| [%] by | | | | | | | |
| weight² | | | | | | | |
| Copovidone ® | 85 | 83 | 83 | 80 | 60 | 60 | 80 |
| VA 64 | | | | | | | |
| (BASF) | | | | | | | |
| PVP | | | | | 20 | 20 | |
| (Kollidon ® | | | | | | | |
| 25, BASF) | | | | | | | |

²Lauroglycol ® FCC is propylene glycol laurate that is available from Gattefosse.
²2-[4-(4-chlorobenzoyl)phenoxy]-2-methyl-propanoic acid, 1-methylethyl ester.

Solid dispersion #1-0 having the composition shown above in Table 11 was prepared as follows. Specifically, crystalline 2-[4-(4-chlorobenzoyl)phenoxy]-2-methyl-propanoic acid, 1-methylethyl ester (Helm, Germany) was blended with Copovidon®. The powder mixture was added manually to the 5 cc micro extruder (DSM Research) and maintained at a temperature of from about 110° C. The powder mixture was mixed in the micro extruder at the elevated temperature for about 2 to about 3 minutes. The melted mass was discharged from the equipment and collected.

Solid dispersions #1-5, 1-8, 1-13, 1-16, 1-17 and 1-21 having the composition shown above in Table 11 were prepared as follows. Each of these solid dispersions (#2-#6) were prepared in the same manner as solid dispersion #1, described above, except that the liquid excipients and polymer were blended with crystalline 2-[4-(4-chlorobenzoyl)phenoxy]-2-methyl-propanoic acid, 1-methylethyl ester (Helm, Germany) prior to extrusion. Extrusion was performed at a temperature of about 110° C. as with solid dispersion #1.

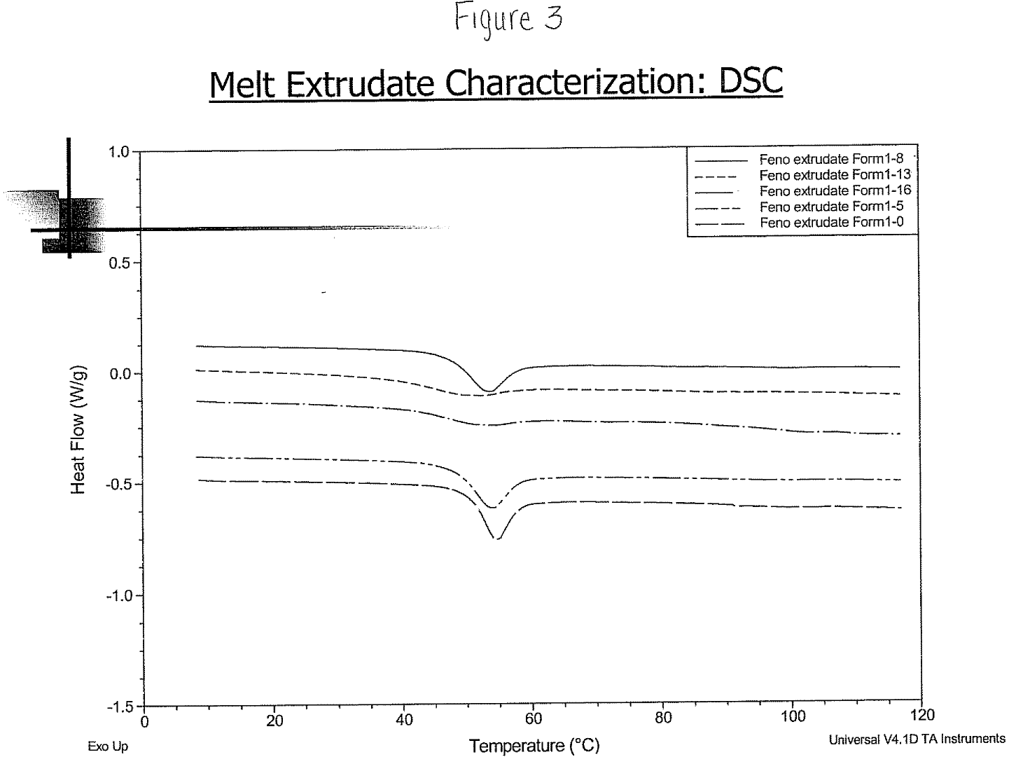

Each of solid dispersions #1-0, 1-5, 1-8, 1-13 and 1-16 were examined by DSC at 10° C. per minute, the technique for which is well known in the art. In all of the samples, DSC analysis indicated that no crystalline 2-[4-(4-chlorobenzoyl)phenoxy]-2-methyl-propanoic acid, 1-methylethyl ester was detected as shown in FIG. 3.

### EXAMPLE 7

### Formation of Suspensions from the Solid Dispersions of Example 6

200 mg of melt extruded solid dispersions 1-0, 1-5, 1-8, 1-13, 1-16, 1-17 and 1-21 prepared as described above in Example 6 were placed into 25 ml of water and stirred with a magnetic stirrer for about 1 hour to form a suspension. FIG. 4 shows three samples of each the suspensions formed by each of solid dispersions 1-0, 1-5, 1-8, 1-13, 1-16, 1-17 and 1-21 after dispersion in the water at various time points. The time points were immediately after formation of the suspension (namely, “initial”), at 6 hours after initial formation, 1 day after initial formation, 3 days after initial formation and 7 days after initial formation.

The particle size of each of the three samples was measured by laser diffraction techniques, the techniques of which are well known to those skilled in the art. Table 12, below, shows the particle size of each of suspensions measured immediately after formation of the suspension (namely, “initial”), 1 day after initial formation, 3 days after initial formation and 7 days after initial formation. The D50 (μm) values are shown below in Table 12.

TABLE 12
Mean Particle Size (D₅₀ μm)

| | Form | Form | Form | Form | Form | Form | Form |
|---|---|---|---|---|---|---|---|
| | 1-0 | 1-5 | 1-8 | 1-13 | 1-16 | 1-17 | 1-21 |
| Run 1 | | | | | | | |
| Initial | 1.7 | 49.2 | 2.5 | 67.9 | 1.8 | 43.6 | 31.2 |
| 1 day | 2.0 | 48.7 | 2.6 | 73.7 | 1.8 | 43.9 | 37.0 |
| 3 days | 1.8 | 54.6 | 3.1 | 81.9 | 1.8 | 50.7 | 43.0 |
| 7 days | 1.7 | 52.3 | 5.1 | 80.7 | 1.8 | 54.5 | 41.7 |
| Run 2 | | | | | | | |
| Initial | 1.8 | 47.9 | 2.7 | 75.5 | 1.8 | 40.0 | 23.3 |
| 1 day | 1.8 | 50.7 | 2.8 | 90.0 | 1.8 | 42.4 | 30.0 |
| 3 days | 1.8 | 54.7 | 2.9 | 98.9 | 1.8 | 47.8 | 35.2 |
| 7 days | 2.0 | 61.6 | 4.1 | 105.9 | 1.8 | 56.2 | 33.4 |
| Run 3 | | | | | | | |
| Initial | 1.7 | 47.1 | 2.8 | 72.4 | 1.7 | 42.5 | 23.9 |
| 1 day | 1.7 | 51.2 | 2.9 | 84.3 | 1.7 | 43.7 | 30.3 |
| 3 days | 1.7 | 53.9 | 3.0 | 95.4 | 1.7 | 46.7 | 32.9 |
| 7 days | 1.7 | 57.2 | 4.2 | 91.3 | 1.8 | 52.4 | 34.7 |

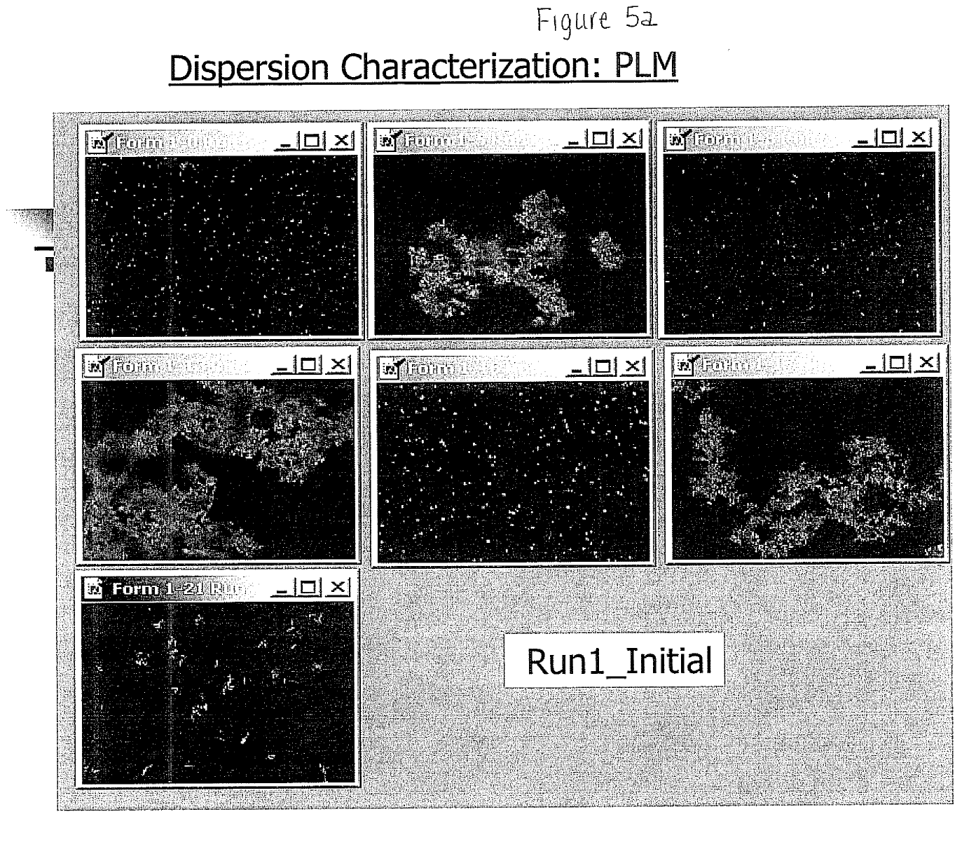

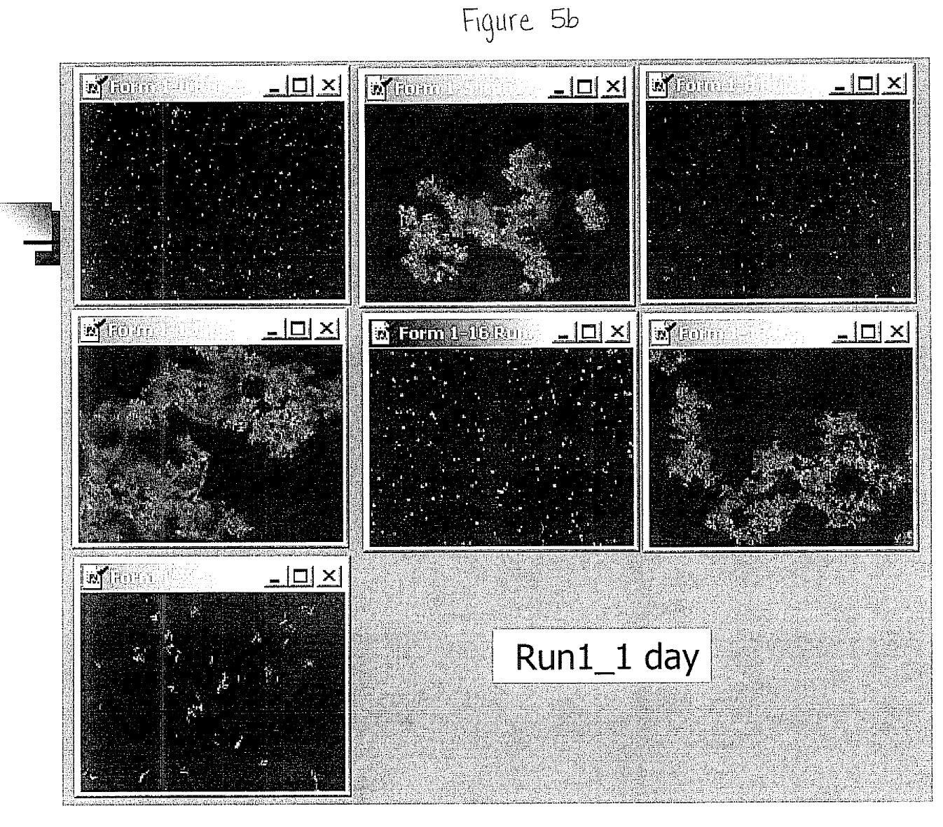

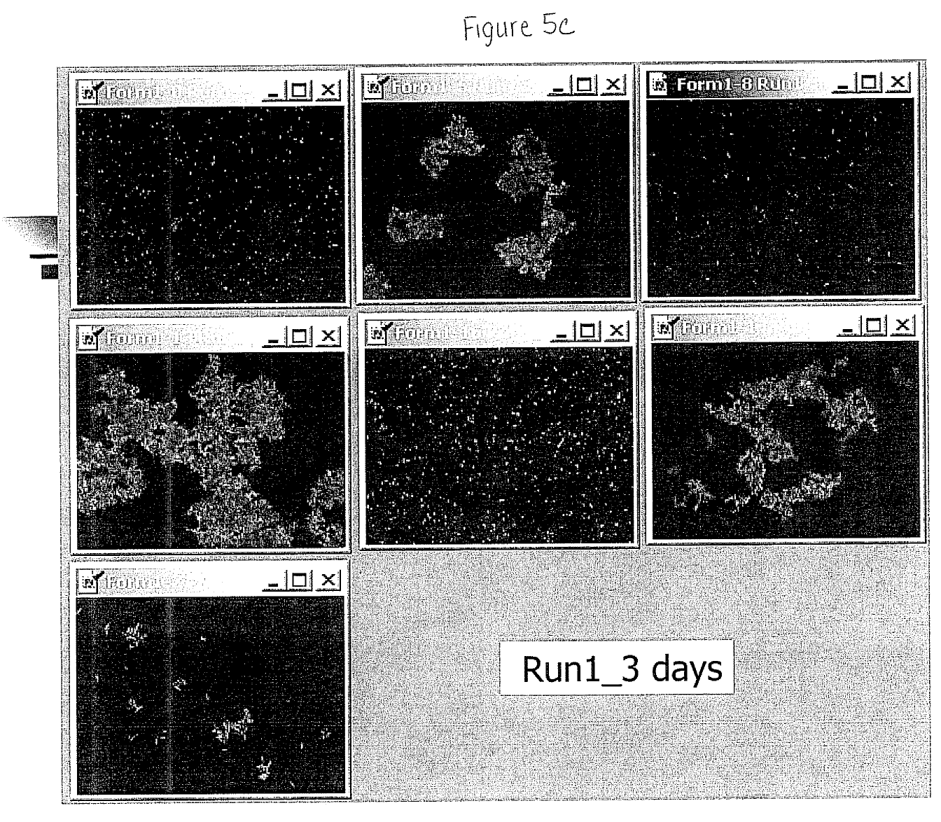

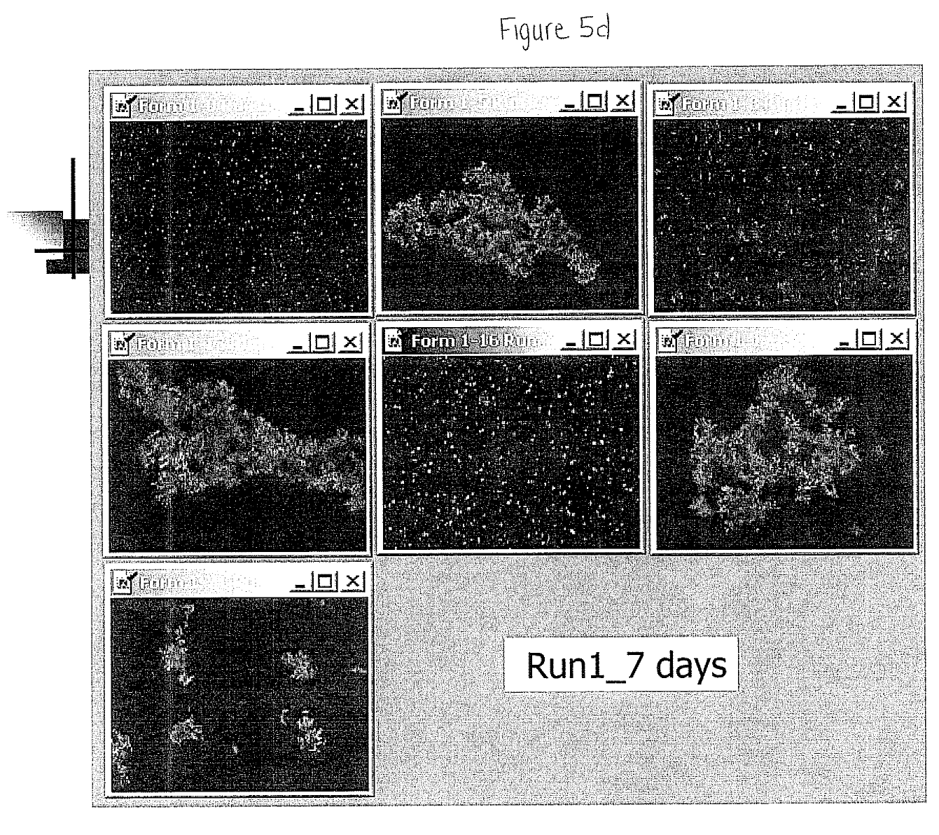

FIG. 5 shows the polarized light microscopy characterization of each of the suspensions as determined immediately after formation (namely, “initial”), 1 day after initial formation, 3 days after initial formation and 7 days after initial formation. The results indicate that the resulting particles contain crystalline 2-[4-(4-chlorobenzoyl)phenoxy]-2-methyl-propanoic acid, 1-methylethyl ester. Therefore, the amorphous 2-[4-(4-chlorobenzoyl)phenoxy]-2-methyl-propanoic acid, 1-methylethyl ester was converted to crystalline 2-[4-(4-chlorobenzoyl)phenoxy]-2-methyl-propanoic acid, 1-methylethyl ester or a mixture of crystalline and amorphous 2-[4-(4-chlorobenzoyl)phenoxy]-2-methyl-propanoic acid, 1-methylethyl ester particles one hour after dispersion in an aqueous medium.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

What is claimed is:

1. An oral pharmaceutical composition comprising at least one active agent and at least one pharmaceutically acceptable polymer, wherein the active agent is primarily amorphous 2-[4-(4-chlorobenzoyl)phenoxy]-2-methyl-propanoic acid,1-methylethyl ester.

2. The composition of claim 1 wherein said composition lacks a significant food effect on oral administration.

3. The composition of claim 1 wherein said composition exhibits improved bioavailability when compared to a 200 mg oral pharmaceutical composition comprising crystalline 2-[4-(4-chlorobenzoyl)phenoxy]-2-methyl-propanoic acid, 1-methylethyl ester.

4. The composition of claim 1 wherein the 2-[4-(4-chlorobenzoyl)phenoxy]-2-methyl-propanoic acid, 1-methylethyl ester is present in the composition in an amount of from about 5 weight percent to about 65 weight percent of the total composition.

5. The composition of claim 4 wherein the 2-[4-(4-chlorobenzoyl)phenoxy]-2-methyl-propanoic acid, 1-methylethyl ester is present in the composition in the amount of from about 10 weight percent to about 50 weight percent of the total composition.

6. The composition of claim 1 wherein said composition further comprises at least one pharmaceutically acceptable surfactant.

7. The composition of claim 1 wherein said composition is in the form of a solid dispersion.

8. The composition of claim 7 wherein said solid dispersion, upon contact with an aqueous medium forms a suspension comprising particles having a size of from about 100 nm to about 10,000 nm.

9. The composition of claim 8 wherein the particles in the suspension comprise crystalline 2-[4-(4-chlorobenzoyl)phenoxy]-2-methyl-propanoic acid, 1-methylethyl ester.

10. The composition of claim 8 wherein the particles in the suspension comprise amorphous 2-[4-(4-chlorobenzoyl)phenoxy]-2-methyl-propanoic acid,1-methylethyl ester.

11. The composition of claim 8 wherein the particles in the suspension comprise a mixture of crystalline and amorphous 2-[4-(4-chlorobenzoyl)phenoxy]-2-methyl-propanoic acid, 1-methylethyl ester.

12. The composition of claim 1 wherein the pharmaceutically acceptable polymer is a nonionic cellulosic polymer.

13. The composition of claim 12 wherein the nonionic cellulosic polymer is selected from the group consisting of methylcellulose, ethyl cellulose, hydroxyethyl cellulose, hydroxypropylcellulose, hydroxypropylmethylcellulose, hydroxypropylmethylcellulose acetate, hydroxyethylmethylcellulose, hydroxyethylcellulose acetate, hydroxyethylethylcellulose and combinations thereof.

14. The composition of claim 1 wherein the pharmaceutically acceptable polymer is selected from the group consisting of: aminoalkyl methacrylate copolymers, carboxylic acid functionalized polymethacrylates, amine-functionalized polymethacrylates, poly(vinyl acetal) diethylaminoacetate, polyvinyl pyrrolidone, polyvinyl alcohol, polyvinyl alcohol/polyvinyl acetate copolymers and combinations thereof.

15. The composition of claim 14 wherein the polymer is selected from the group consisting of polyvinyl pyrrolidone, polyvinyl alcohol/polyvinyl acetate copolymers and combinations thereof.

16. The composition of claim 1 wherein the pharmaceutically acceptable polymer is copovidone.

17. The composition of claim 1 wherein the pharmaceutically acceptable polymer is selected from the group consisting of polyethyleneoxide polyethylene glycol/polypropylene glycol copolymers, polyethylene/polyvinyl alcohol copolymers, dextran, pullulan, acacia, tragacanth, sodium alginate, propylene glycol alginate, agar powder, gelatin, starch, processed starch, glucomman, chitosan and combinations thereof.

18. The composition of claim 1 wherein the pharmaceutically acceptable polymer is present in the composition in an amount of from about 20 weight percent to about 95 weight percent.

19. The composition of claim 18 wherein the pharmaceutically acceptable polymer is present in the composition in an amount of from about 30 weight percent to about 75 weight percent.

20. The composition of claim 6 wherein the pharmaceutically acceptable surfactant has a hydrophile-lipophile balance value from about 1 to about 20.

21. The composition of claim 6 wherein the pharmaceutically acceptable surfactant is present in the composition in an amount of from about 0.5 weight percent to about 20 weight percent.

22. The composition of claim 21 wherein the pharmaceutically acceptable surfactant is present in the composition in an amount of from about 1 weight percent to about 8 weight percent.

23. The composition of claim 6 wherein the pharmaceutically acceptable surfactant is selected from the group consisting of: triglycerides of caprylic/capric acid, propylene glycol laurate, glyceryl and polyethylene glycol esters, sorbitan monooleate, sorbitan monolaurate, mono or diglycerides of caprylic/capric acid in glycerol, sorbitan sesquioleate, polyoxyethylene (2) oleyl ether, polyoxypropylene 15 stearyl ether, unsaturated polyglycolyzed glycerides, glyceryl monolinoleate, decaglyceryl decaoleate, triisostearin polyethylene glycol 6 esters, triglyceryl monoleate, glyceryl monooleate, sorbide dioleate, polyoxyethylene castor wax, polyglycolysed glycerides, polyglycolysed glycerides, saturated C₈-C₁₀ polyglycolysed glycerides, polyoxyethlene (20) sorbitan monooleate, polyoxyethylene (20) sorbitan trioleate, copolymers of propylene oxide and ethylene oxide, polyoxyl 35 castor oil, palm kernelamide, polyoxyethylene 4 lauryl ether, polyoxyethylene (20) isohexadecyl ether, sorbitan monolaurate, alcohol ethoxylate, polyoxyethylene 80 sorbitan monolaurate, hexaglyceryl dioleate, polysorbate 80, sucrose laurate, quaternary ammonium salt, polyoxyethylene sorbitol hexaoleate, caprylic/capric acid partial glyceride-6 EO, polyglyceryl 4 oleate, and combinations thereof.

24. The composition of claim 23 wherein the pharmaceutically acceptable surfactant is selected from the group consisting of: triglycerides of caprylic/capric acid, glyceryl and polyethylene glycol esters, sorbitan monolaurate, polyoxyethylene (20) sorbitan trioleate, polyoxyl 35 hydrogenated castor oil and combinations thereof.

25. The composition of claims 24 wherein the pharmaceutically acceptable surfactant is triglycerides of caprylic/capric acid and polyoxyl 35 hydrogenated castor oil.

26. The composition of claim 6 wherein the pharmaceutically acceptable surfactant is distillated acetylated monoglycerides, distilled acetylated monoglycerides, propylene glycol and mono or dicaprylate, polyoxypropylene (15) stearyl alcohol, glyceryl tricaprylate/caprate, olive oil, caprylic/capric triglycerides, sesame oil, oleyl alcohol and combinations thereof.

27. The composition of claim 1 wherein the composition further comprises at least one solubility-enhancing agent.

28. The composition of claim 27 wherein the solubility-enhancing agent is present in the composition in the amount of from about 1 weight percent to about 40 weight percent.

29. The composition of claim 28 wherein the solubility-enhancing agent is present in the composition in the amount of from about 1 weight percent to about 10 weight percent.

30. The composition of claim 29 wherein the solubility-enhancing agent is at least one surfactant, at least one pH control agent, glycerides, partial glycerides, glyceride derivatives, polyoxyethylene and polypropylene esters and copolymers, sorbitan esters, polyoxyethylene sorbitan esters, carbonate salts, alkyl sulfonates, cyclodextrins and combinations thereof.

31. The composition of claim 30 wherein the pH control agent is a buffer, organic acid, organic acid salts, organic bases, inorganic bases and combinations thereof.

32. The composition of claim 1 wherein the composition further comprises a at least one coating, tableting aid, water-soluble polymer, filler, binder, pigment, distintegrant, antioxidant, lubricant, flow aid, flavorant.

33. A method of treating dyslipidemia in a subject in need of treatment thereof, the method comprising the step of:
administering to said subject a therapeutically effective amount of the pharmaceutically acceptable composition of claim 1.

34. A method of treating dyslipoproteinemia in a subject in need of treatment thereof, the method comprising the step of:
administering to said subject a therapeutically effective amount of the pharmaceutically acceptable composition of claim 1.

35. An oral pharmaceutical composition comprising at least one active agent and at least one pharmaceutically acceptable polymer, wherein the active agent is primarily amorphous 2-[4-(4-chlorobenzoyl)phenoxy]-2-methyl-propanoic acid, 1-methylethyl ester that upon contact with aqueous media forms a suspension.

36. The composition of claim 8, wherein the particles do not agglomerate for a period of from about fifteen (15) minutes to about seven (7) days.
